(19) United States
(12) Patent Application Publication
(10) Pub. No.: US 2026/0137691 A1
(43) Pub. Date: May 21, 2026

(54) WB3 FOR ANESTHESIA AND TREATING PAIN

(71) Applicant: President and Fellows of Harvard College, Cambridge, MA (US)

(72) Inventors: Haleh Fotowat, Cambridge, MA (US); Donald E. Ingber, Cambridge, MA (US); Megan Sperry, Cambridge, MA (US); Joel D. Moore, Cambridge, MA (US)

(21) Appl. No.: 19/365,779

(22) Filed: Oct. 22, 2025

Related U.S. Application Data
(60) Provisional application No. 63/711,084, filed on Oct. 23, 2024.

(51) Int. Cl. A61K 31/5375; A61P 23/00; A61P 29/02

(52) U.S. Cl. CPC A61K 31/5375; A61P 23/00; A61P 29/02

(57) ABSTRACT

Provided herein is a compound of Formula (1)(WB3), or a pharmaceutically acceptable salt thereof, compositions thereof, and methods and uses for inducing anesthesia and/or treating pain.

### RELATED APPLICATIONS

This application claims priority under 35 U.S.C. § 119(e) to U.S. Provisional patent application, U.S. Ser. No. 63/711,084, filed Oct. 23, 2024, which is incorporated herein by reference in its entirety.

### GOVERNMENT SUPPORT

This invention was made with government support under W911NF-24-2-0112 awarded by U.S. Army Research Office (ARO). The government has certain rights in the invention.

### BACKGROUND

General anesthesia is typically induced in an operating room at a hospital or in a dedicated anesthetic room adjacent, such as an intensive care unit, emergency department, or ambulance, to allow for controlled induction of anesthesia, vital-sign monitoring, physiological maintenance, and/or possible intervention by medical professionals. Accordingly, general anesthesia cannot easily be induced at the site of a disaster due to the lack of anesthetic compounds or cocktails that can be used safely outside of a hospital facility. Thus, there is a large unmet need for anesthetics that can be used for inducing anesthesia away from a hospital setting, while keeping the autonomic nervous system and physiological functions stable, such that there would be no need for specialized medical equipment for their stabilization.

### SUMMARY

The present disclosure stems from the recognition that drugs that induce anesthesia without the need for (a) extensive monitoring, (b) life-support equipment, and/or (c) highly trained medical expertise in a traditional hospital setting would have great benefit in interventions at a point of injury (e.g., a battlefield or car crash). As such, pharmacological induction of anesthesia by physiological slowing offers a new therapeutic approach for trauma management and enhancing patient survival in remote and low-resource locations. The present disclosure also contemplates the use of such drugs for treating pain (e.g., pain caused by a disease, disorder, or condition or injury) without inducing anesthetic effects by lowering the dose of the drug administered to the subject.

Accordingly, disclosed herein are compounds, compositions, and methods that slow metabolism and mimic states normally induced by hypothermia, hibernation, or torpor, and are useful for inducing anesthesia in a subject in need thereof. In some embodiments, the compounds induce anesthetic effects rapidly and are safely reversible.

In one aspect, disclosed are methods of inducing anesthesia in a subject in need thereof, the method comprising administering a compound of Formula (1):

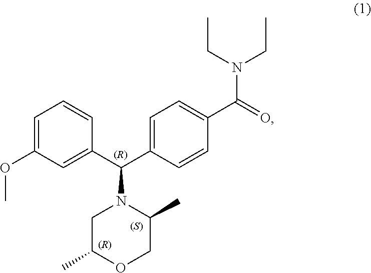

or a pharmaceutically acceptable salt, solvate, hydrate, polymorph, co-crystal, tautomer, stereoisomer, isotopically labeled derivative, or prodrug thereof to the subject. In certain embodiments, the compound is administered in a pharmaceutical composition comprising one or more pharmaceutically acceptable excipients. In certain embodiments, the compound is administered at a dose sufficient to achieve a desired anesthetic effect. In certain embodiment, the desired anesthetic effect is selected from the group consisting of general anesthesia, sedation, tranquilization, immobility, amnesia, analgesia, unconsciousness, and autonomic quiescence. In certain embodiments, the desired anesthetic effect is general anesthesia.

In another aspect, disclosed are methods of treating pain (e.g., a painful condition) in a subject in need thereof, the methods comprising administering a compound of Formula (1), or a pharmaceutically acceptable salt, solvate, hydrate, polymorph, co-crystal, tautomer, stereoisomer, isotopically labeled derivative, or prodrug thereof, or a pharmaceutical composition comprising the compound of Formula (1). In certain embodiments, administering the compound of Formula (1) does not induce general anesthesia, sedation, tranquilization, immobility, amnesia, unconsciousness, and/or autonomic quiescence.

In another aspect, disclosed are pharmaceutical compositions comprising a compound of Formula (1), or a pharmaceutically acceptable salt thereof, and a pharmaceutically acceptable excipient.

The present disclosure also contemplates the use of the compounds and compositions described herein for inducing anesthesia in a subject in need thereof and/or treating pain in a subject in need thereof.

In another aspect, disclosed are kits comprising the compound of Formula (1), or a pharmaceutically acceptable salt thereof, or a pharmaceutical composition comprising the compound of Formula (1); and instructions for administering the compound, the pharmaceutically acceptable salt thereof, or the pharmaceutical composition to a subject.

The details of certain embodiments of the invention are set forth in the Detailed Description of Certain Embodiments, as described below. Other features, objects, and advantages of the invention will be apparent from the Definitions, Examples, Figures, and Claims.

### BRIEF DESCRIPTION OF THE DRAWINGS

FIGS. 1A-1E show an experimental setup for verifying lack of consciousness and nociception. FIG. 1A shows the protocol developed to screen state of the art (SOA) anesthetics and “metabolic modulating compounds” (MMCs) using mechanosensory (via physical “taps”) and UV-activated noxious stimuli (Optovin). FIG. 1B shows pain stimulus. FIG. 1C shows tap stimulus to access consciousness. Optovin concentration and stimulus duration were optimized to elicit a roust, plate-wide response to stimuli. As expected, there was a high degree of inter-individual variability in responsiveness to either stimulation type. FIG. 1D and FIG. 1E shows response (movement patterns) of tadpoles after receiving stimuli. FIG. 1D shows response of tadpoles after 5 second of UV-stimulation. FIG. 1E shows response of tadpoles after tap stimulation.

FIGS. 2A-2B demonstrate that both ketamine- and WB3-treated tadpoles display reduced responsiveness to painful stimuli, with the WB3 group showing better recovery. FIG. 2A shows ketamine-treated tadpole response to Optovin stimulation. FIG. 2B shows WB3-treated tadpole response to Optovin stimulation. “A” traces show tadpole response to Optovin stimulation prior to any treatment of anesthetics (without ketamine or WB3). “B” traces show tadpole response to Optovin stimulation after treatment of anesthetics (FIG. 2A, with ketamine (500 uM); FIG. 2B, with WB3 (100 uM)). “C” trace shows tadpole response after 3 hours of recovery. FIGS. 2A-2B shows that WB3 is as effective as ketamine at a lower dose, and that the group treated with WB3 has overall better recovery.

FIGS. 3A-3B show that both ketamine- and WB3-treated tadpoles display reduced responsiveness to physical/mechanical stimuli (e.g., physical taps to the plate, “tap stimulus”), with the WB3 group showing better recovery. FIG. 3A shows the tadpole response to tap stimulus in the ketamine-treated group. FIG. 2B shows WB3 group and the response to tap stimulus. “A” traces show tadpole response to tap stimulus prior to any treatment of anesthetics (without ketamine or WB3). “B” traces show tadpole response to tap stimulus after treatment of anesthetics (FIG. 3A, with ketamine (500 uM); FIG. 3B, with WB3 (100 uM)). “C” trace shows tadpole response after 3 hours of recovery. FIGS. 3A-3B show that WB3 is as effective as ketamine at a lower dose, and that the group treated with WB3 has overall better recovery.

FIGS. 4A-4C show a method for quantifying pain response amplitude. First the average duration of a response was estimated by calculating the average activity across all 24 tadpoles in each treatment group and finding the time between stimulus onset and the end of response, which was defined as the time the response declined to 33% of its peak. The area under the response curve (AUC) was then calculated for each tadpole during this average time window. FIG. 4B shows ketamine response AUC distribution by treatment. FIG. 4C shows WB3 response AUC distribution by treatment.

FIGS. 5A-5C show dose-dependent suppression of movement and subsequent recovery in ketamine- and WB3-treated tadpoles. FIG. 5A shows ketamine-treated group. FIG. 5B shows WB3-treated group. Ketamine treatment at concentrations lower than 500 uM were effective at inducing immobility, and a full recovery was observed within an hour after wash out. FIG. 5C shows that tadpoles treated with up to 200 uM WB3 show full recovery.

FIG. 6 compares movement suppression and subsequent recovery of tadpoles treated with WB3, ketamine, etomidate, and dexmedetomidine. FIG. 6 shows treatment with 100 uM of ketamine, etomidate, and dexmedetomidine was effective at inducing immobility in tadpoles (treatment phase). Tadpoles treated with 100 uM ketamine were more active than those treated with 100 uM WB3 at the end of the 1.5-hour treatment. The time course of induction for ketamine, etomidate, and dexmedetomidine was faster than that of WB3 at the same concentration. Recovery from ketamine occurred quickly upon transfer to regular media. WB3 showed a faster recovery profile compared to etomidate. Tadpoles treated with dexmedetomidine did not regain normal movement.

FIGS. 7A-7B show WB3 produces distinct transcriptional effects compared to vehicle. FIG. 7A shows snap frozen and Trizol-prepared samples exhibit different transcriptional profiles. FIG. 7B shows WB3 produces distinct transcriptional effects compared to vehicle.

FIGS. 8A-8C show WB3 produced distinct transcriptional effects compared to vehicle exposure. FIG. 8A shows a volcano plot of WB3 transcriptional effects compared to vehicle exposure of snap frozen and Trizol-prepared samples. FIG. 8B shows WB3 produced distinct transcriptional effects compared to vehicle exposure, in particular downregulated pathways. FIG. B. shows, broadly, that signaling processes, including receptor and ligand activities, are suppressed with WB3 treatment. This includes immune and hormonal signaling. Some families of hormones were downregulated, including IGF-1 (responsible for glucose transport). IGF-1 is also decreased by sevoflurane anesthesia (Xie et al., J Neuro Physiology 2021). Aspects of the immune system are suppressed by WB3, including genes involved in B cell, macrophage, and neutrophil activities. The effect of different anesthetics on neutrophil function is diverse (Meier & Nizet Anesthesia & Analgesia 2019). FIG. 8C shows the pathways that were upregulated in WB3-treated tadpoles. These pathways include genes like birc7s, which is an inhibitor of apoptosis and serpin55, which is predicted to enable serine-type endopeptidase inhibitor activity. Endopeptidases are enzymes that break peptide bonds, therefore, upregulation of the activity of the inhibitor, results in reduced activity of these enzymes.

FIG. 9 compares movement suppression and subsequent recovery of tadpoles treated with WB3, ketamine, etomidate, and propofol. FIG. 9 shows treatment with ketamine, etomidate and propofol was effective at inducing immobility in tadpoles. WB3 at 100 uM and 200 uM show dose dependent induction and recovery. Results indicate WB3 was as effective as ketamine, etomidate, and propofol. WB3 at 100 uM showed comparable recovery to ketamine (at 500 uM). WB3 at 200 uM showed a slower, step-like recovery.

FIGS. 10A-10B show dose-dependent suppression of movement and subsequent recovery in WB3- and ketamine-treated tadpoles. FIG. 10A shows the dose response of tadpoles treated with WB3. FIG. 10B shows the dose response of tadpoles treated with either ketamine or WB3. FIG. 10A shows WB3 at higher doses was more effective at inducing suppression of movement. FIG. 10B shows WB3 at lower doses shows synergy with ketamine.

FIG. 11 shows movement suppression and subsequent recovery of tadpoles treated with various MMCs in mobility assay.

FIG. 12 shows responsiveness to painful shock stimuli was suppressed in tadpoles treated with WB3 in a dose-dependent manner.

FIG. 13 shows response suppression of tap stimulus (“mechanosensory”) and subsequent response recovery of tadpoles treated with various MMCs.

FIG. 14 shows response suppression to pain stimuli (via Optovin exposure) and subsequent response recovery of tadpoles treated with various MMCs.

FIG. 15 shows response suppression to tap stimulus (“mechanosensory”) and subsequent response recovery of tadpoles treated with WB3, ketamine, etomidate, and propofol.

FIG. 16 shows response suppression to pain stimulus (via Optovin exposure) and subsequent response recovery of tadpoles treated with WB3, ketamine, etomidate, and propofol.

FIG. 17 shows response suppression to electric shock and subsequent response recovery of tadpoles treated with WB3, ketamine, etomidate, and propofol.

FIG. 18 shows pharmacokinetic/pharmacodynamic study of WB3 in tadpoles. Internal concentration of WB3 mirrors the observed effects on movement from 100 μM dosing.

FIGS. 19A-19C show cell-based assays that demonstrate delta opioid receptor activity is shut down in WB3. FIG. 19A shows that WB3 does not activate delta opioid receptors. FIG. 19B shows that WB3 does not bind to Kappa opioid receptors. FIG. 19C shows that WB3 does not bind to Mu opioid receptors.

FIGS. 20A-20B compare movement suppression and movement recovery of tadpoles treated with either a cytochrome inhibitor (talarozole) or with WB3.

FIGS. 21A-21B compare movement suppression and movement recovery of tadpoles treated with either a cytochrome inhibitor or with WB3.

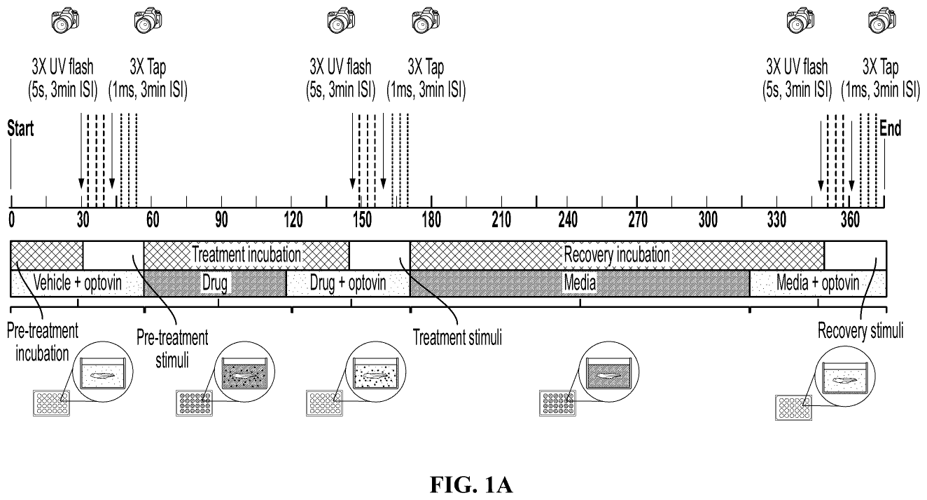

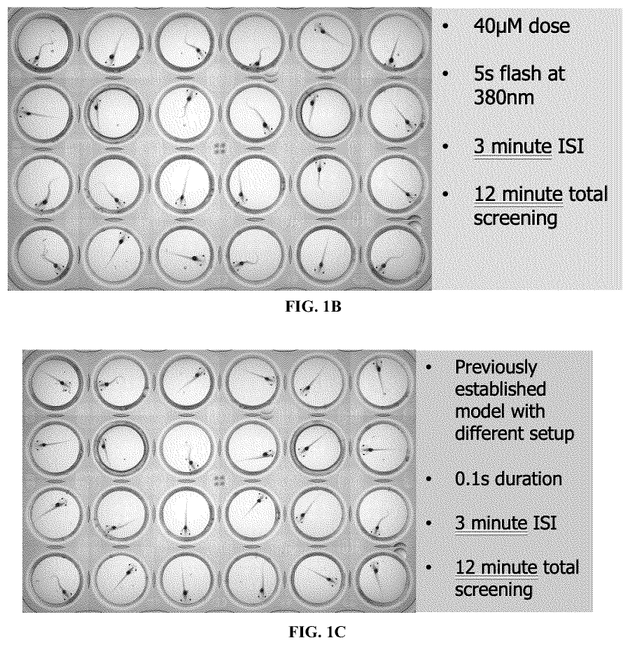

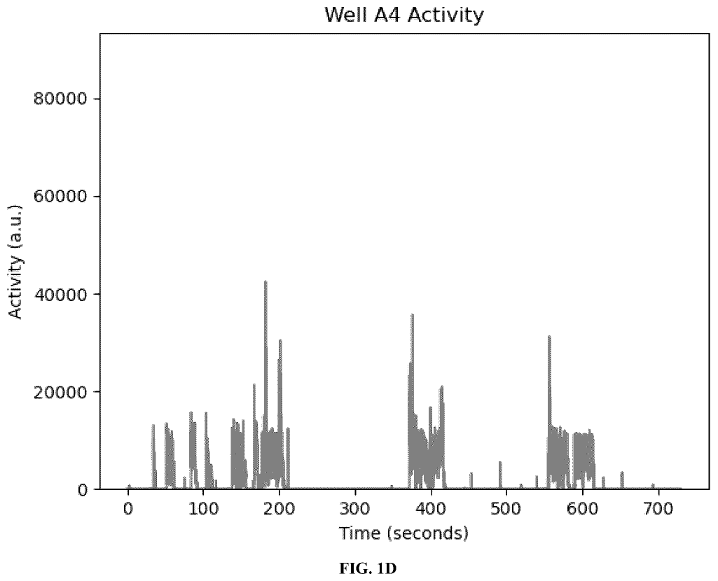

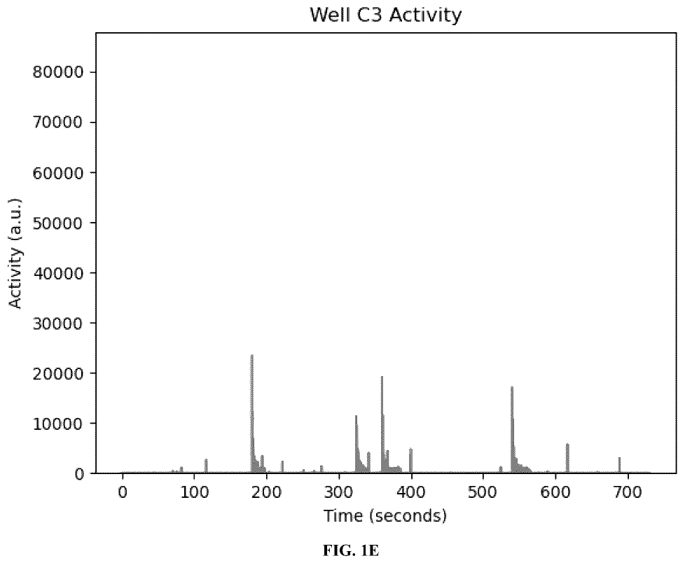

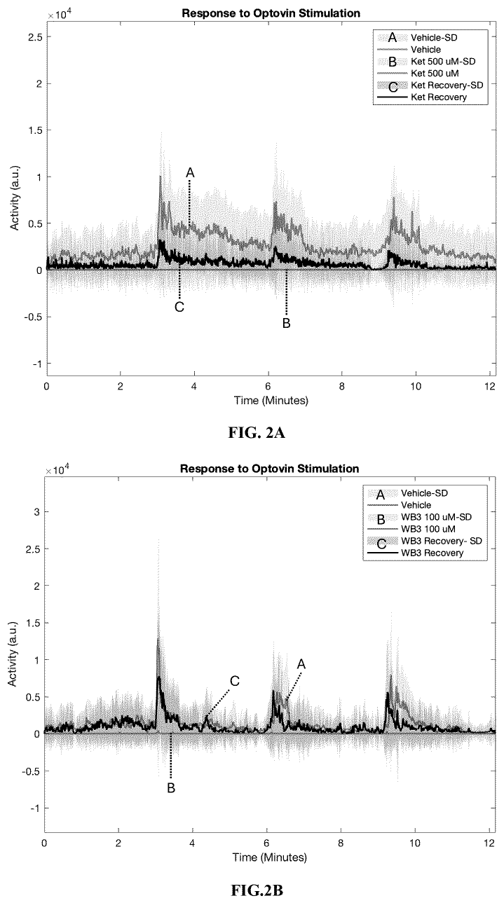

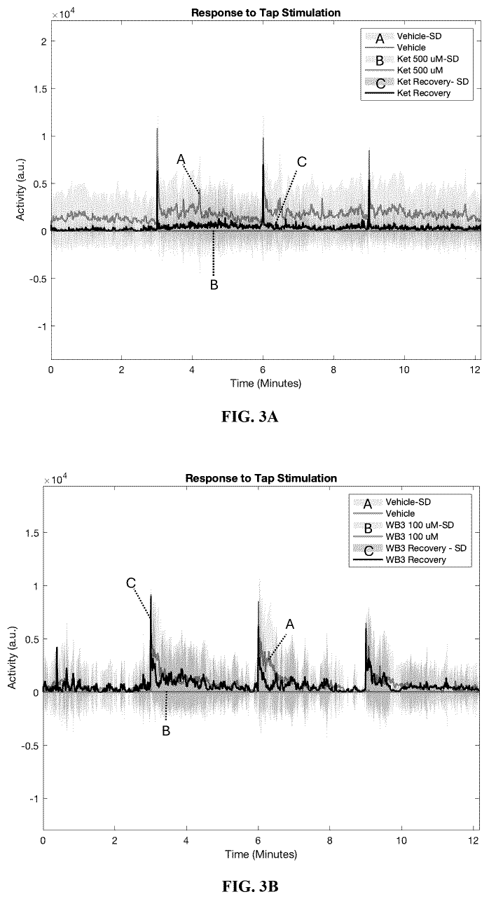

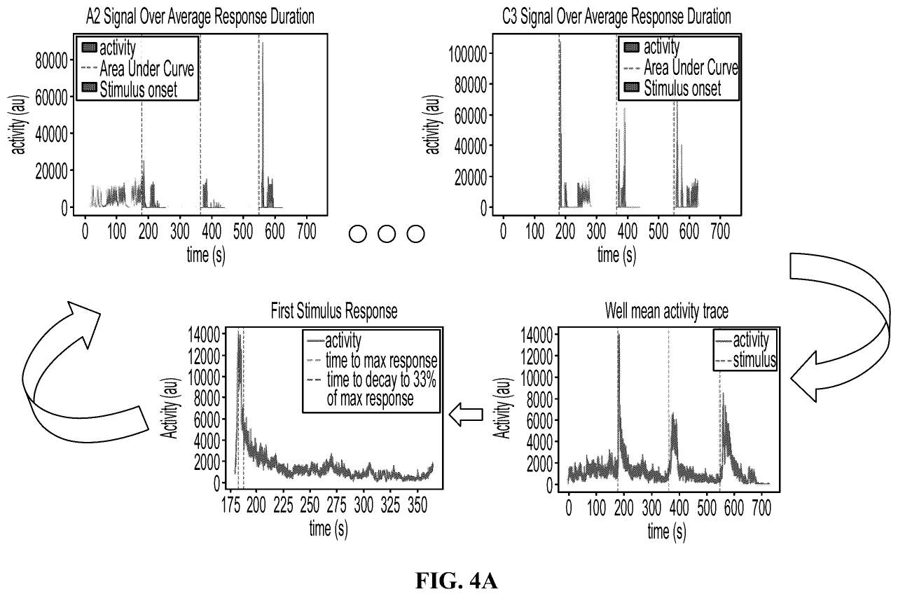

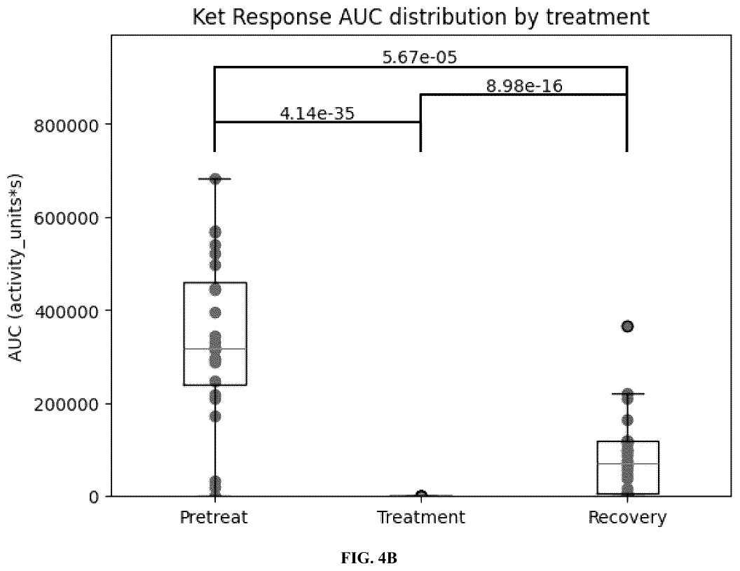

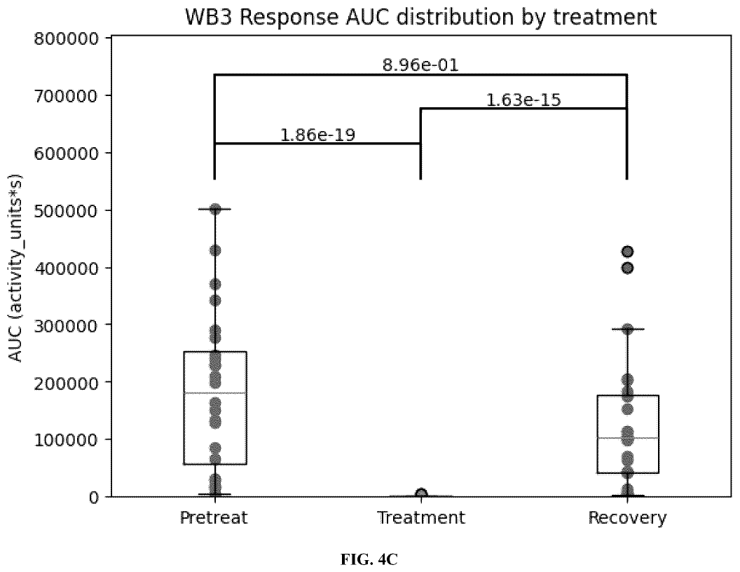

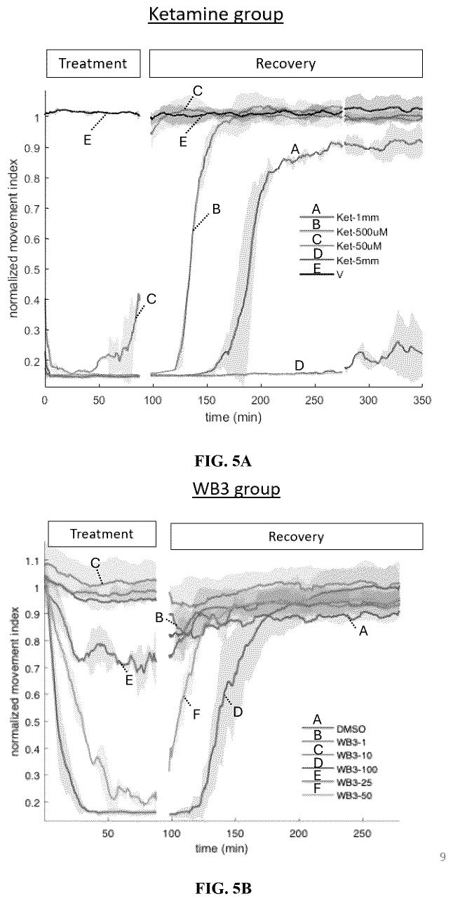

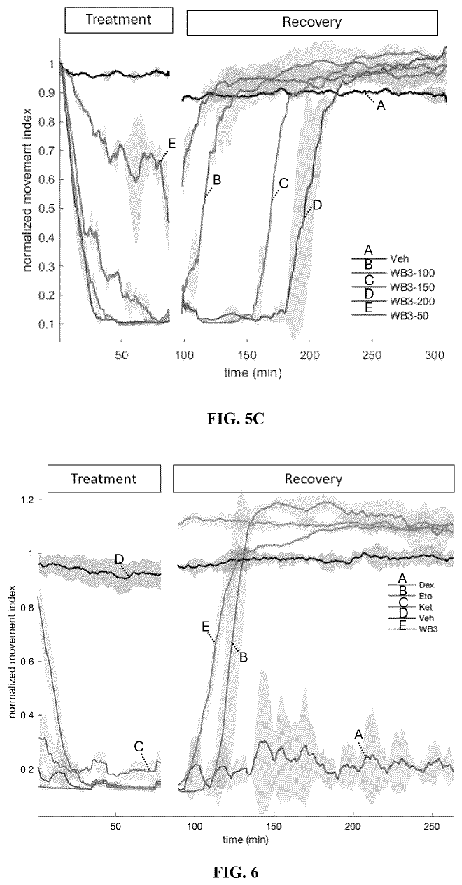

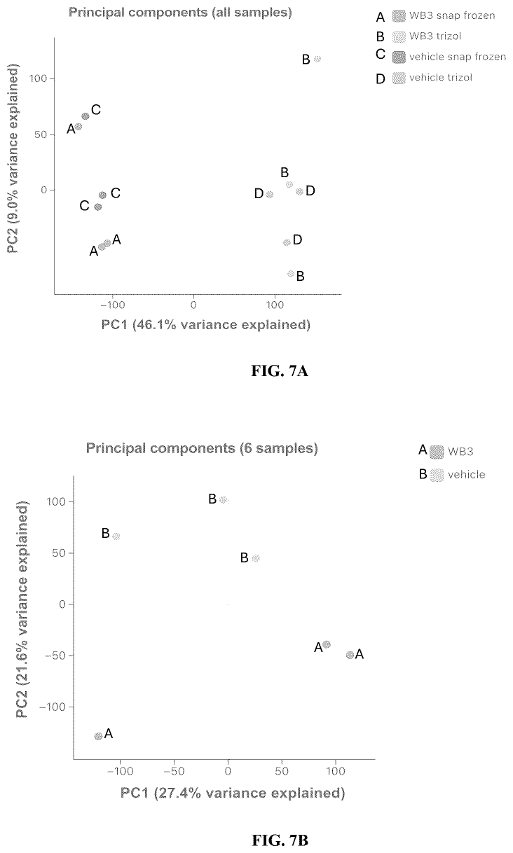

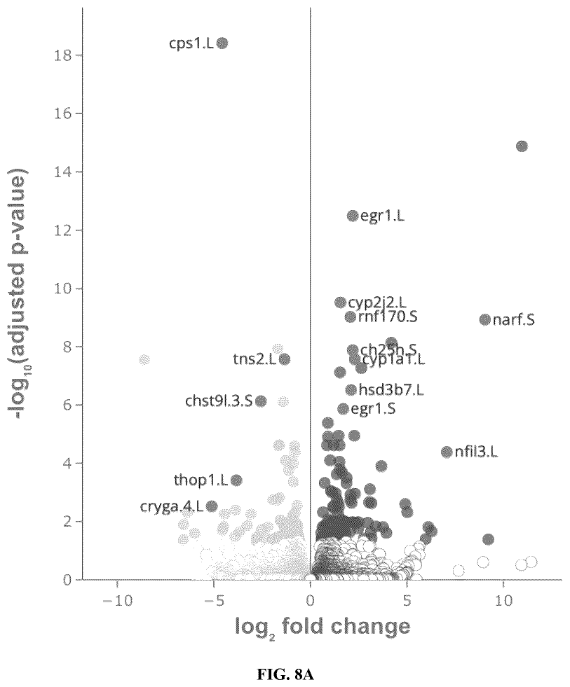

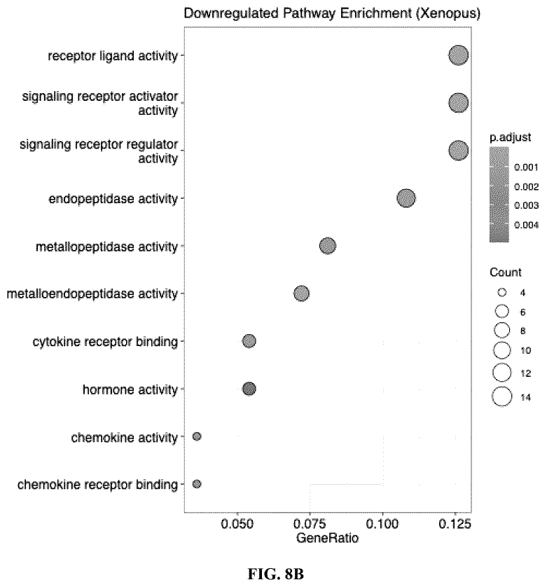

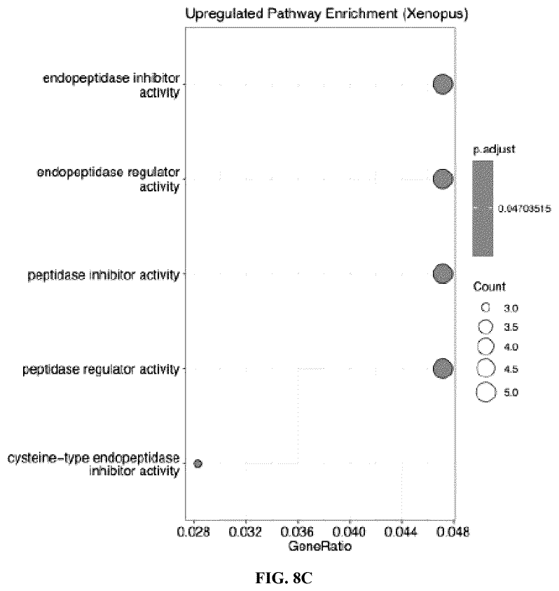

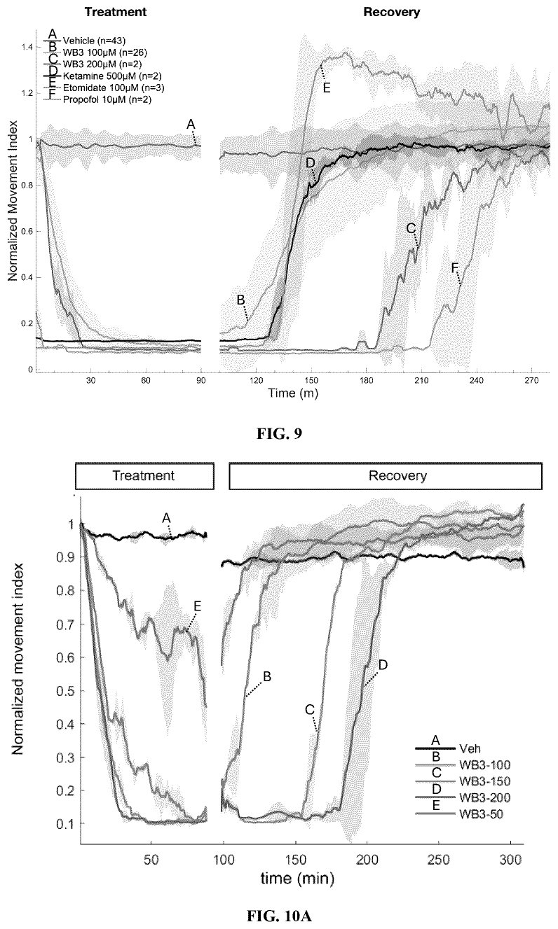

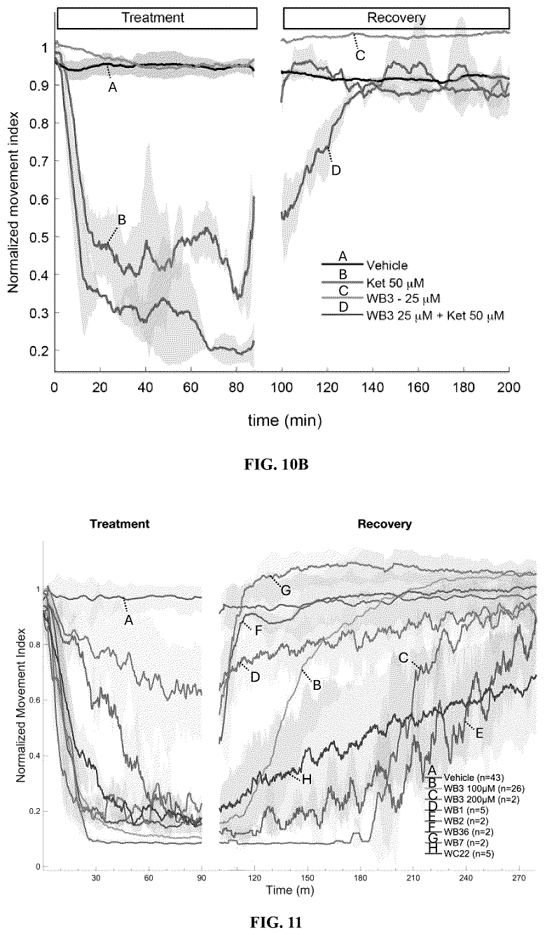

### DEFINITIONS

As used herein, the term “salt” refers to any and all salts, and encompasses pharmaceutically acceptable salts.

A “compound of Formula (1)” or “WB3” refers to the compound having the following formula:

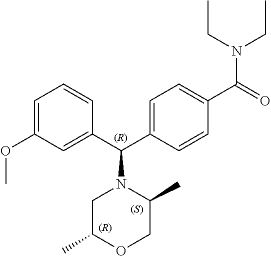

Preparation of WB3 is disclosed in U.S. provisional application, U.S. Ser. No. 63/591,821, which is incorporated herein by reference in its entirety.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁₋₈ alkyl, C₂₋₈ alkenyl, C₂₋₈ alkynyl, aryl, C₇₋₁₂ substituted aryl, and C₇₋₁₂ arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. The subject may also be a plant. In certain embodiments, the plant is a land plant. In certain embodiments, the plant is a non-vascular land plant. In certain embodiments, the plant is a vascular land plant. In certain embodiments, the plant is a seed plant. In certain embodiments, the plant is a cultivated plant. In certain embodiments, the plant is a dicot. In certain embodiments, the plant is a monocot. In certain embodiments, the plant is a flowering plant. In some embodiments, the plant is a cereal plant, e.g., maize, corn, wheat, rice, oat, barley, rye, or millet. In some embodiments, the plant is a legume, e.g., a bean plant, e.g., soybean plant. In some embodiments, the plant is a tree or shrub.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “tissue” refers to any biological tissue of a subject (including a group of cells, a body part, or an organ) or a part thereof, including blood and/or lymph vessels, which is the object to which a compound, particle, and/or composition of the disclosure is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment.” “treat.” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for inducing anesthesia and/or treating pain.

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

The terms “biologic,” “biologic drug,” and “biological product” refer to a wide range of products such as vaccines, blood and blood components, allergenics, somatic cells, gene therapy, tissues, nucleic acids, and proteins. Biologics may include sugars, proteins, or nucleic acids, or complex combinations of these substances, or may be living entities, such as cells and tissues. Biologics may be isolated from a variety of natural sources (e.g., human, animal, microorganism) and may be produced by biotechnological methods and other technologies.

The term “small molecule” or “small molecule therapeutic” refers to molecules, whether naturally occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

The term “anesthesia” refers to the loss of sensation with or without loss of consciousness. Anesthesia is considered a medical treatment that prevents patients from feeling pain during procedures like surgery, certain screening and diagnostic tests, tissue sample removal (e.g., skin biopsies), and dental work, and medicines used for anesthesia, are called anesthetics. General anesthesia affects the whole body, making patients unconscious and unable to move. Monitored sedation is like general anesthesia in that it relaxes the body and may induce sleep; however, in monitored sedation, patients are still conscious. Regional anesthesia numbs pain and sensation to only the part of the body that needs it, such as an arm, a leg. With regional anesthetics, patients stay conscious. Local anesthesia affects only a small part of the body. For example, this type of anesthetic is used to block pain to a single tooth during a dental procedure or to a part of the skin that needs stitches. As with regional anesthesia, patients who receive local anesthesia remain conscious. In certain embodiments, the desired anesthetic effect is selected from the group consisting of general anesthesia, sedation, tranquilization, immobility, amnesia, analgesia, unconsciousness, and autonomic quiescence.

The term “pain” refers to “a painful condition.”

A “painful condition” includes, but is not limited to, neuropathic pain (e.g., peripheral neuropathic pain), central pain, deafferentation pain, chronic pain (e.g., chronic nociceptive pain, and other forms of chronic pain such as post-operative pain, e.g., pain arising after hip, knee, or other replacement surgery), pre-operative pain, stimulus of nociceptive receptors (nociceptive pain), acute pain (e.g., phantom and transient acute pain), noninflammatory pain, inflammatory pain, pain associated with cancer, wound pain, burn pain, postoperative pain, pain associated with medical procedures, pain resulting from pruritus, painful bladder syndrome, pain associated with premenstrual dysphoric disorder and/or premenstrual syndrome, pain associated with chronic fatigue syndrome, pain associated with pre-term labor, pain associated with withdrawal symptoms from drug addiction, joint pain, arthritic pain (e.g., pain associated with crystalline arthritis, osteoarthritis, psoriatic arthritis, gouty arthritis, reactive arthritis, rheumatoid arthritis or Reiter's arthritis), lumbosacral pain, musculo-skeletal pain, headache, migraine, muscle ache, lower back pain, neck pain, toothache, dental/maxillofacial pain, visceral pain and the like. One or more of the painful conditions contemplated herein can comprise mixtures of various types of pain provided above and herein (e.g. nociceptive pain, inflammatory pain, neuropathic pain, etc.). In some embodiments, a particular pain can dominate. In other embodiments, the painful condition comprises two or more types of pains without one dominating. A skilled clinician can determine the dosage to achieve a therapeutically effective amount for a particular subject based on the painful condition.

In certain embodiments, the painful condition is neuropathic pain. The term “neuropathic pain” refers to pain resulting from injury to a nerve. Neuropathic pain is distinguished from nociceptive pain, which is the pain caused by acute tissue injury involving small cutaneous nerves or small nerves in muscle or connective tissue. Neuropathic pain typically is long-lasting or chronic and often develops days or months following an initial acute tissue injury. Neuropathic pain can involve persistent, spontaneous pain as well as allodynia, which is a painful response to a stimulus that normally is not painful. Neuropathic pain also can be characterized by hyperalgesia, in which there is an accentuated response to a painful stimulus that usually is trivial, such as a pin prick. Neuropathic pain conditions can develop following neuronal injury and the resulting pain may persist for months or years, even after the original injury has healed. Neuronal injury may occur in the peripheral nerves, dorsal roots, spinal cord or certain regions in the brain. Neuropathic pain conditions include, but are not limited to, diabetic neuropathy (e.g., peripheral diabetic neuropathy); sciatica; non-specific lower back pain; multiple sclerosis pain; carpal tunnel syndrome, fibromyalgia; HIV-related neuropathy: neuralgia (e.g., post-herpetic neuralgia, trigeminal neuralgia); pain resulting from physical trauma (e.g., amputation; surgery, invasive medical procedures, toxins, burns, infection), pain resulting from cancer or chemotherapy (e.g., chemotherapy-induced pain such as chemotherapy-induced peripheral neuropathy), and pain resulting from an inflammatory condition (e.g., a chronic inflammatory condition). Neuropathic pain can result from a peripheral nerve disorder such as neuroma; nerve compression: nerve crush, nerve stretch or incomplete nerve transection; mononeuropathy or polyneuropathy. Neuropathic pain can also result from a disorder such as dorsal root ganglion compression; inflammation of the spinal cord; contusion, tumor or hemisection of the spinal cord; tumors of the brainstem, thalamus or cortex; or trauma to the brainstem, thalamus or cortex.

The symptoms of neuropathic pain are heterogeneous and are often described as spontaneous shooting and lancinating pain, or ongoing, burning pain. In addition, there is pain associated with normally non-painful sensations such as “pins and needles” (paresthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia), or an absence of or deficit in selective sensory pathways (hypoalgesia).

In certain embodiments, the painful condition is non-inflammatory pain. The types of non-inflammatory pain include, without limitation, peripheral neuropathic pain (e.g., pain caused by a lesion or dysfunction in the peripheral nervous system), central pain (e.g., pain caused by a lesion or dysfunction of the central nervous system), deafferentation pain (e.g., pain due to loss of sensory input to the central nervous system), chronic nociceptive pain (e.g., certain types of cancer pain), noxious stimulus of nociceptive receptors (e.g., pain felt in response to tissue damage or impending tissue damage), phantom pain (e.g., pain felt in a part of the body that no longer exists, such as a limb that has been amputated), pain felt by psychiatric subjects (e.g., pain where no physical cause may exist), and wandering pain (e.g., wherein the pain repeatedly changes location in the body).

In certain embodiments, the painful condition is inflammatory pain. In certain embodiments, the painful condition (e.g., inflammatory pain) is associated with an inflammatory condition and/or an immune disorder.

### DETAILED DESCRIPTION OF CERTAIN EMBODIMENTS

Provided herein are methods for inducing anesthesia in a subject in need thereof, comprising administering a compound of Formula (1):

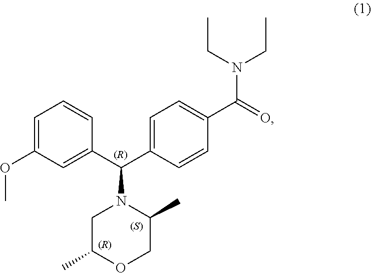

or a pharmaceutically acceptable salt, solvate, hydrate, polymorph, co-crystal, tautomer, stereoisomer, isotopically labeled derivative, or prodrug thereof to the subject.

In certain embodiments, the compound is administered in a pharmaceutical composition comprising one or more pharmaceutically acceptable excipients.

In certain embodiments, the compound of Formula (1), or pharmaceutical composition comprising the compound of Formula (1), is administered orally, topically, intramuscularly, intravenously, parenterally, or by inhalation. In certain embodiments, the compound of Formula (1), or pharmaceutical composition comprising the compound of Formula (1), is administered orally, topically, intramuscularly, intravenously, or parenterally.

In certain embodiments, the compound of Formula (1) is administered at a dose sufficient to achieve a desired anesthetic effect. In certain embodiments, the desired anesthetic effect is selected from the group consisting of general anesthesia, sedation, tranquilization, immobility, amnesia, analgesia, unconsciousness and autonomic quiescence. In certain embodiments, the desired anesthetic effect is general anesthesia.

In another embodiment, the present disclosure contemplates methods for inducing anesthesia in a subject, wherein inducing anesthesia in a subject comprises physiological slowing in the subject. In certain embodiments, the physiological slowing comprises slowing of metabolic processes in the subject. In certain embodiments, the physiological slowing is reversible in the subject. In certain embodiments, reversing physiological slowing comprises restoration of mobility and/or consciousness in the subject.

In another embodiment, the present disclosure contemplates the use of any of the methods disclosed herein for treating a subject that is undergoing surgery and/or a medical procedure. In certain embodiments, the subject is in pain and/or injured. In certain embodiments, the subject is a mammal (e.g., a human). In certain embodiments, the subject is a human.

In certain embodiments, the methods described herein inducing general anesthesia while keeping the autonomic nervous system and physiological functions stable, such that there would be no need for specialized medical equipment for their stabilization. The present disclosure contemplates the use of these methods for inducing general anesthesia at a site of injury, for example, a battlefield.

In another aspect, the present disclosure describes methods of treating pain in a subject in need thereof, comprising administering a compound of Formula (1):

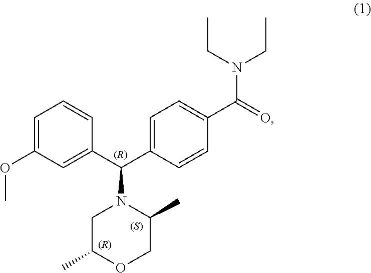

or a pharmaceutically acceptable salt, solvate, hydrate, polymorph, co-crystal, tautomer, stereoisomer, isotopically labeled derivative, or prodrug thereof to the subject. The methods may comprise treating pain a subject in need thereof. In certain embodiments, the present disclosure provides a method for suppressing pain in a subject in need thereof.

In certain embodiments, the compound of Formula (1) is administered in a pharmaceutical composition comprising one or more pharmaceutically acceptable excipients. In certain embodiments, the compound of Formula (1), or pharmaceutical composition comprising the compound of Formula (1), is administered orally, topically, intramuscularly, intravenously, parenterally, or by inhalation. In certain embodiments, the compound of Formula (1), or pharmaceutical composition comprising the compound of Formula (1), is administered orally, topically, intramuscularly, intravenously, or parenterally.

In certain embodiments, provided herein are methods of treating pain (a painful condition), by administering a compound of Formula (1), or a pharmaceutical composition comprising a compound of Formula (1), the method comprising administering an effective amount of a compound of Formula (1) or composition as provided herein to a subject in need thereof.

In certain embodiments, the painful condition is neuropathic pain. In some embodiments, the painful condition is chronic pain. In some embodiments, the painful condition is due to nerve damage, injury, spinal cord injury, pain associated with spinal disc degeneration, radiculitis, radiculopathy, neuritis, inflammation, thermal or mechanical hyperalgesia, thermal or mechanical allodynia, iatrogenic neuralgia, diabetic pain, pain arising from irritable bowel or other internal organ disorders, endometriosis pain, phantom limb pain, complex regional pain syndromes, fibromyalgia, low back pain, cancer pain, pain arising from infection, inflammation or trauma to peripheral nerves or the central nervous system, multiple sclerosis pain, entrapment pain, pain from HIV infection, herpesvirus infection, and pain from infections. In some embodiments, the painful condition is due to nerve damage. In some embodiments, the painful condition is due to injury. In some embodiments, the painful condition is due to spinal cord injury. In some embodiments, the painful condition is due to pain associated with spinal disc degeneration. In some embodiments, the painful condition is due to radiculitis. In some embodiments, the painful condition is due to radiculopathy. In some embodiments, the painful condition is due to neuritis. In some embodiments, the painful condition is due to inflammation. In some embodiments, the painful condition is due to thermal or mechanical hyperalgesia. In some embodiments, the painful condition is due to thermal or mechanical allodynia. In some embodiments, the painful condition is due to iatrogenic neuralgia. In some embodiments, the painful condition is due to diabetic pain. In some embodiments, the painful condition is due to pain arising from irritable bowel or other internal organ disorders. In some embodiments, the painful condition is due to endometriosis pain. In some embodiments, the painful condition is due to phantom limb pain. In some embodiments, the painful condition is due to complex regional pain syndromes. In some embodiments, the painful condition is due to fibromyalgia. In some embodiments, the painful condition is due to low back pain. In some embodiments, the painful condition is due to cancer pain. In some embodiments, the painful condition is due to pain arising from infection. In some embodiments, the painful condition is due to inflammation or trauma to peripheral nerves or the central nervous system. In some embodiments, the painful condition is due to multiple sclerosis pain. In some embodiments, the painful condition is due to entrapment pain. In some embodiments, the painful condition is due to pain from HIV infection. In some embodiments, the painful condition is due to herpesvirus infection. In some embodiments, the painful condition is due to pain from infection.

In certain embodiments, pain is nociceptive pain, neuropathic pain, or a combination thereof. In certain embodiments, pain is acute pain, chronic pain, mild pain, moderate pain, or severe pain. In certain embodiments, the present disclosure contemplates methods of treating pain in a subject in need thereof, wherein administering the compound of Formula (1) at a lower dose does not induce general anesthesia, sedation, tranquilization, immobility, amnesia, unconsciousness, and/or autonomic quiescence. In certain embodiments, the subject is a mammal (e.g., a human). In certain embodiments, the subject is a human.

In certain embodiments, the compound of Formula (1) is provided in a concentration of equal to or less than 500 μM, equal to or less than 400 μM, equal to or less than 300 μM, equal to or less than 200 μM, equal to or less than 100 μM, equal to or less than 75 μM, equal to or less than 50 μM, equal to or less than 25 μM, or equal to or less than 10 μM. In certain embodiments, the compound of Formula (1) is provided in a concentration of 200 μM. In certain embodiments, the compound of Formula (1) is provided in a concentration of 100 μM. In certain embodiments, the compound of Formula (1) is provided in a concentration of 50 μM.

In certain embodiments, the compound of Formula (1) is not a delta opioid receptor agonist. In certain embodiments, the compound of Formula (1) inhibits the delta opioid receptor with an IC₅₀ of greater than 10 nM, greater than 50 nM, greater than 100 nM, greater than 500 nM, greater than 1,000 nM, or greater than 10,000 nM.

In certain embodiments, the methods of the disclosure comprise administering to the subject an effective amount of a compound of the disclosure (e.g., a compound of Formula (1)), or a pharmaceutically acceptable salt, co-crystal, tautomer, stereoisomer, solvate, hydrate, polymorph, isotopically enriched derivative, or prodrug, or composition thereof. In certain embodiments, the methods of the disclosure comprise administering to the subject an effective amount of a compound of the disclosure (e.g., a compound of Formula (1)), or a pharmaceutically acceptable salt thereof.

In some embodiments, the effective amount is a therapeutically effective amount. In some embodiments, the effective amount is a prophylactically effective amount.

In certain embodiments, the methods comprise administering a compound of the disclosure, or a pharmaceutically acceptable salt, co-crystal, tautomer, stereoisomer, solvate, hydrate, polymorph, isotopically enriched derivative, or prodrug, or composition thereof, or a pharmaceutical composition comprising a compound of the disclosure, to a subject. In certain embodiments, the methods comprise administering a compound of the disclosure, or a pharmaceutically acceptable salt, co-crystal, tautomer, stereoisomer, solvate, hydrate, polymorph, isotopically enriched derivative, or prodrug, or composition thereof, or a pharmaceutical composition comprising a compound of the disclosure, to a cell. In certain embodiments, the methods comprise administering a compound of the disclosure, or a pharmaceutically acceptable salt, co-crystal, tautomer, stereoisomer, solvate, hydrate, polymorph, isotopically enriched derivative, or prodrug, or composition thereof, or a pharmaceutical composition comprising a compound of the disclosure, to a subject.

In certain embodiments, the present disclosure provides a compound for use in inducing anesthesia. In certain embodiments, the present disclosure provides a compound of Formula (1) for use in inducing physiological slowing. In certain embodiments, the physiological slowing comprises slowing of metabolic processes. In certain embodiments, the physiological slowing is reversible. In certain embodiments, reversing physiological slowing comprises restoration of mobility and/or consciousness in the subject.

In certain embodiments, the present disclosure provides a compound of Formula (1) for use in inducing a desired anesthetic effect, for example, general anesthesia, sedation, tranquilization, immobility, amnesia, analgesia, unconsciousness, and autonomic quiescence. In certain embodiments, the desired anesthetic effect is general anesthesia. In certain embodiments, the desired anesthetic effect is sedation. In certain embodiments, the desired anesthetic effect is tranquilization. In certain embodiments, the desired anesthetic effect is immobility. In certain embodiments, the desired anesthetic effect is amnesia. In certain embodiments, the desired anesthetic effect is analgesia. In certain embodiments, the desired anesthetic effect is unconsciousness. In certain embodiments, the desired anesthetic effect is autonomic quiescence.

In certain embodiments, the present disclosure provides a compound of Formula (1) for use in inducing a desired anesthetic effect, for example, general anesthesia while keeping the autonomic nervous system and physiological functions stable, such that there would be no need for specialized equipment for their stabilization (e.g., respiration of a subject is not impaired).

In certain embodiments, the present disclosure provides a compound for use in treating pain in a subject. In certain embodiments, the present disclosure provides a compound of Formula (1) for use in suppressing pain in a subject. In certain embodiments, a compound of Formula (1) is administered at a dose that allows for the treatment of pain (e.g., a pain condition) without inducing anesthetic effect, for example, general anesthesia, sedation, tranquilization, immobility, amnesia, analgesia, unconsciousness, and autonomic quiescence.

In certain embodiments, the present disclosure provides a compound for use in the manufacture of a medicament for inducing anesthesia. In certain embodiments, the present disclosure provides a compound for use in the manufacture of a medicament for inducing physiological slowing. In certain embodiments, the physiological slowing comprises slowing of metabolic processes. In certain embodiments, the physiological slowing is reversible. In certain embodiments, reversing physiological slowing comprises restoration of mobility and/or consciousness in the subject.

In certain embodiments, the present disclosure provides a compound for use in the manufacture of a medicament for treating pain. In certain embodiments, the present disclosure provides a compound for use in the manufacture of a medicament for suppressing pain in a subject.

In certain embodiments, the subject being treated is an animal. The animal may be of either sex and may be at any stage of development. In certain embodiments, the subject is a mammal. In certain embodiments, the subject being treated is a human. In certain embodiments, the subject is a non-human animal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Certain methods described herein may comprise administering one or more additional pharmaceutical agent(s) in combination with the compounds described herein. The additional pharmaceutical agent(s) may be administered at the same time as a compound of the disclosure (e.g., a compound of Formula (1)), or at different times than a compound of the disclosure (e.g., a compound of Formula (1)). For example, a compound of the disclosure (e.g., a compound of Formula (1)) and any additional pharmaceutical agent(s) may be on the same dosing schedule or different dosing schedules. All or some doses of a compound of the disclosure (e.g., a compound of Formula (1)) may be administered before all or some doses of an additional pharmaceutical agent, after all or some does an additional pharmaceutical agent, within a dosing schedule of an additional pharmaceutical agent, or a combination thereof. The timing of administration of a compound of the disclosure (e.g., a compound of Formula (1)) and additional pharmaceutical agents may be different for different additional pharmaceutical agents. In certain embodiments, the additional pharmaceutical agent comprises an agent useful for inducing anesthesia. In certain embodiments, the additional pharmaceutical agent is useful for inducing physiological slowing. In certain embodiments, the additional pharmaceutical agent comprises an agent useful for treating pain. In certain embodiments, the additional pharmaceutical agent comprises an agent useful for suppressing pain in a subject.

### Pharmaceutical Compositions, Kits, and Administration

The present disclosure provides pharmaceutical compositions comprising a compound of Formula (1) (referred to herein as “WB3”), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (1), or a pharmaceutically acceptable salt thereof, and a pharmaceutically acceptable excipient.

In certain embodiments, a compound of Formula (1) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for inducing anesthesia in a subject. In certain embodiments, the effective amount is an amount effective for inducing physiological slowing in a subject, biological sample, or cell. In certain embodiments, the effective amount is an amount effective for treating pain in a subject. In certain embodiments, the effective amount is an amount effective for suppressing pain in a subject.

Pharmaceutical compositions described herein can be prepared by any method known in the art of pharmacology. In general, such preparatory methods include the steps of bringing the composition comprising a compound of Formula (1) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as, for example, one-half or one-third of such a dosage.

The compound and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical, mucosal, nasal, bucal, sublingual; by intratracheal instillation, bronchial instillation, and/or inhalation; and/or as an oral spray, nasal spray, and/or aerosol.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In certain embodiments, the subject is an animal. The animal may be of either sex and may be at any stage of development. In certain embodiments, the subject is a mammal. In certain embodiments, the subject described herein is a human. In certain embodiments, the subject is a non-human animal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for inducing anesthesia in a subject. In certain embodiments, the kits are useful for inducing physiological slowing in a subject. In certain embodiments, the kits are useful for treating pain in a subject, for example, by suppressing pain in a subject in need thereof. In certain embodiment, the kits are useful for treating pain in a subject in need thereof without inducing anesthesia.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, a kit described herein may include one or more additional pharmaceutical agents described herein as a separate composition. In certain embodiments, the instructions comprise administering a compound of Formula (1), or a pharmaceutically acceptable salt thereof, or a pharmaceutical composition comprising a compound of Formula (1), to mammalian tissue (e.g., human tissue).

### EXAMPLES

### Behavioral Experiments

For each compound to be tested, the following procedures were performed:

### Pre-Treatment Measurements

Tadpoles were placed individually in wells of 24 well plates in a 40 μM Optovin solution (in DMSO) and allowed to acclimate for 30 minutes. Optovin is a UV activatable TRPA channel agonist, which acts as painful stimulus upon UV activation (Kokel et al 2013, Wee et al 2019). Using Ramona optics microscope. 5-s pulses of UV were presented to activate Optovin while recording the behavior of the tadpoles. The UV stimulus was presented 3 times, with an interstimulus interval (ISI) of 3 minutes to avoid habituation. Tadpoles were next presented with 3 mechanosensory tap stimuli at ISI of 3 minutes in the same setup and their behavior was recorded.

### Post-Treatment Measurements

Tadpoles were transferred to the wells of a new 24 well plate filled with solution containing compound of interest (compound of Formula (1)). The total duration of treatment was 1.5 hours. In the last 30 minutes of the treatment, the tadpoles were transferred to a new 24 well plate filled with the solution containing the compound of interest (compound of Formula (1))+Optovin. This was done to avoid potential toxicity of Optovin on tadpoles over long treatment times. After a total of 1.5 hours of treatment, the tadpoles were presented with 3 pulses of UV at ISI=3 min followed by 3 tap stimuli, following the exact pattern presented in pre-treatment, and their behavior was recorded.

### Post-Recovery Measurements

Tadpoles were transferred to the wells of a new 24 well plate filled with solution regular media (MMR) and allowed to recover for a total of 3 hours. After 2.5 hours in MMR, they were transferred to a new 24 well plate with MMR+Optovin and allowed to acclimate and recover for 30 more minutes. After a total of 3 hours recovery, the tadpoles were presented with 3 pulses of UV at ISI=3 min followed by 3 tap stimuli, following the exact pattern presented in pre-treatment, and their behavior was recorded.

### Data Analysis

An activity measure was extracted from the video recordings of each tadpole. This was done by the analysis software of the microscope through quantifying changes observed in the consecutive video frames. Average and standard deviation plots were generated using a custom MATLAB code by averaging the activity measure across the 24 tadpoles per treatment condition. Area under the activity curve for each tadpole was used to perform statistical comparisons across conditions. Kruskalwallis test with multiple comparisons was used to calculate statistical significance of the difference across groups.

### Transcriptomics

At the end of the treatment period (1.5 h), individual tadpoles were placed in Eppendorf PCR tubes, snap frozen in liquid nitrogen, and submitted to a CRO (Novogene) for RNA extraction and sequencing.

### Ligand Binding Assay

The compounds were submitted to Eurofins for in-vitro pharmacology studies in binding and enzyme activity assays. Compound binding was calculated as a % inhibition of the binding of a ligand specific for each target. Compound enzyme inhibition effect was calculated as a % inhibition of control enzyme activity.

### SUMMARY

Study Design: WB3 was tested at 1.0E−05 M.

Measurements: Compound binding was calculated as a % inhibition of the binding of a ligand specific for each target. Compound enzyme inhibition effect was calculated as a % inhibition of control enzyme activity.

Results: Results showing an inhibition or stimulation higher than 50% are considered to represent significant effects of the test compounds. Such effects were observed here and are listed in the following tables.

WB3 did not show binding to known targets of state of the art anesthetics, namely NMDA receptors (ketamine), GABA-A receptors (propofol, etomidate), and adrenergic receptors (dexmedetomidine).

TABLE 1

| Assay | 1.0E−05M* |
|---|---|
| CB₂(h) (agonist radioligand) | 50.3% |
| delta (DOP) (h) (agonist radioligand) | 75.8% |
| kappa (h) (KOP) (agonist radioligand) | 64.5% |
| μ (MOP) (h) (agonist radioligand) | 55.4% |
| GR (h) (agonist radioligand) | 54.7% |
| CDK2/CyclinA Human CMGC Kinase | 91.3% |
| Enzymatic Radiometric [Km ATP] | |

*Concentration of WB3

### Compounds

TABLE 2
Test Compounds

| | Batch | | | Received | Stock |
|---|---|---|---|---|---|
| Compound ID | Number | FW | Purity | Form | solution |
| 100074212-1 | 3 | 410.56 | 100.0 | Powder | 1.E−02M |
| (WB3) | | | | | DMSO |

FW: Formula Weight - MW: Molecular Weight

### Reference Compounds

In each experiment and if applicable, the respective reference compound was tested concurrently with WB3, and the data were compared with historical values determined at Eurofins. The experiment was accepted in accordance with Eurofins validation. Standard Operating Procedure.

### Results

### In Vitro Pharmacology: Binding Assays

TABLE 3
Test Compound Results

| | % | | | |
|---|---|---|---|---|
| | Inhibition | | | |
| | of Control | % of Control | | |
| | Specific | Specific Binding | | |
| Assay | Binding | 1st | 2nd | Mean |
| Sodium Channel Site2 (Non-selective) Rat | 19 | 79.1 | 82.5 | 80.8 |
| Ion Channel Batrachotoxin Mass | | | | |
| Spectrometry Binding | | | | |
| Glutamate (Non-Selective) Rat Ion Channel | 0 | 97.9 | 102.9 | 100.4 |
| Glycine (Strychnine-Insensitive) Mass | | | | |
| Spectrometry Binding | | | | |
| Cav1.2 (L-type) Rat Calcium Ion Channel | 28 | 87.3 | 57.6 | 72.4 |
| (Diltiazem Site) Mass Spectrometry Binding | | | | |
| GABAB(B1b/B2) Human GABA B GPCR Mass | 12 | 91.3 | 84.5 | 87.9 |
| Spectrometry Binding (Antagonist Ligand) | | | | |
| Assay, Cerep | | | | |
| A1 (h) (agonist radioligand) | 5 | 89 | 100.3 | 94.6 |
| A2A (h) (agonist radioligand) | 13 | 87.1 | 86.7 | 86.9 |
| A2B (h) (antagonist radioligand) | 7 | 84.3 | 102 | 93.2 |
| A3 (h) (agonist radioligand) | 20 | 88.3 | 71 | 79.7 |
| alpha1A (h) (antagonist radioligand) | 8 | 93.5 | 91.3 | 92.4 |
| alpha1B (h) (antagonist radioligand) | 1 | 95.7 | 102.5 | 99.1 |
| alpha2A (h) (antagonist radioligand) | −6 | 106.5 | 105.8 | 106.2 |
| alpha2B (h) (antagonist radioligand) | −8 | 106.1 | 110.5 | 108.3 |
| alpha2C (h) (antagonist radioligand) | 1 | 99 | 98 | 98.5 |
| beta1 (h) (agonist radioligand) | −3 | 107.8 | 98.2 | 103 |
| beta2 (h) (antagonist radioligand) | −11 | 106.2 | 115.2 | 110.7 |
| Beta3(h) (antagonist radioligand) | 0 | 103.9 | 96.3 | 100.1 |
| AT1 (h) (antagonist radioligand) | 15 | 81.3 | 89.3 | 85.3 |
| AT2 (h) (agonist radioligand) | −1 | 100.6 | 101.9 | 101.3 |
| APJ (apelin) (h) (agonist radioligand) | −2 | 103.3 | 101.2 | 102.2 |
| BZD (central) (agonist radioligand) | −7 | 111.5 | 102.2 | 106.8 |
| BB3 (h) (agonist radioligand) | −1 | 99.3 | 102.7 | 101 |
| B2 (h) (agonist radioligand) | 2 | 99.1 | 97.6 | 98.3 |
| CB2 (h) (agonist radioligand) | 50 | 51.6 | 47.9 | 49.7 |
| CB1 (h) (agonist radioligand) | 23 | 78.3 | 76.4 | 77.4 |
| CCK1 (CCKA) (h) (agonist radioligand) | 2 | 99.9 | 96.4 | 98.2 |
| CCK2 (CCKB) (h) (agonist radioligand) | 14 | 83 | 89.1 | 86 |
| CRF1 (h) (agonist radioligand) | 6 | 95.8 | 93.1 | 94.4 |
| D1 (h) (antagonist radioligand) | 5 | 96 | 94.8 | 95.4 |
| D2S (h) (agonist radioligand) | −5 | 110 | 99.4 | 104.7 |
| D3 (h) (antagonist radioligand) | 2 | 91.7 | 104.3 | 98 |
| ETA (h) (agonist radioligand) | 1 | 103.4 | 94.8 | 99.1 |
| ETB (h) (agonist radioligand) | −10 | 103.3 | 116.5 | 109.9 |
| GABAA1 (h) (alpha1, beta2, gamma2) (agonist | −3 | 101.9 | 104.1 | 103 |
| radioligand) | | | | |
| GABAA (Non-Selective) Rat Ion Channel | 23 | 67.2 | 87.8 | 77.5 |
| Picrotoxinin Mass Spectrometry Binding | | | | |
| Antagonist Ligand | | | | |
| glucagon (h) (agonist radioligand) | −6 | 103 | 109.5 | 106.3 |
| AMPA (agonist radioligand) | 12 | 80.2 | 96.7 | 88.4 |
| kainate (agonist radioligand) | 10 | 93.3 | 86.7 | 90 |
| NMDA (antagonist radioligand) | −3 | 103.2 | 103.4 | 103.3 |
| mGluR1 (agonist radioligand) | 0 | 104.3 | 96.4 | 100.4 |
| mGluR5 (h) (agonist radioligand) | −5 | 104.7 | 105.9 | 105.3 |
| TNF-alpha (h) (agonist radioligand) | −9 | 99.9 | 118.2 | 109.1 |
| CCR2 (h) (agonist radioligand) | −12 | 112.6 | 111.2 | 111.9 |
| H1 (h) (antagonist radioligand) | −4 | 102.8 | 105.6 | 104.2 |
| H2 (h) (antagonist radioligand) | −5 | 103.8 | 106.1 | 104.9 |
| H3 (h) (agonist radioligand) | 3 | 100 | 94.7 | 97.4 |
| H4 (h) (agonist radioligand) | 14 | 87.8 | 84.6 | 86.2 |
| CysLT1 (LTD4) (h) (agonist radioligand) | −1 | 97.4 | 103.9 | 100.6 |
| MCH1 (h) (agonist radioligand) | −13 | 106.4 | 120.2 | 113.3 |
| MC1 (agonist radioligand) | 24 | 76.8 | 74.8 | 75.8 |
| MC3 (h) (agonist radioligand) | −18 | 109 | 127.6 | 118.3 |
| MC4 (h) (agonist radioligand) | 7 | 98.3 | 88.5 | 93.4 |
| MT1 (ML1A) (h) (agonist radioligand) | 18 | 84.6 | 78.4 | 81.5 |
| MT3 (ML2) (agonist radioligand) | 26 | 71.1 | 77.8 | 74.4 |
| MAO-A (antagonist radioligand) | 4 | 97.6 | 94.8 | 96.2 |
| motilin (h) (agonist radioligand) | 7 | 91.1 | 95.7 | 93.4 |
| M1 (h) (antagonist radioligand) | −2 | 102.6 | 100.6 | 101.6 |
| M2 (h) (antagonist radioligand) | 11 | 85.9 | 91.3 | 88.6 |
| M3 (h) (antagonist radioligand) | 5 | 94.1 | 95.9 | 95 |
| M4 (h) (antagonist radioligand) | 0 | 99.1 | 101.5 | 100.3 |
| NK1 (h) (agonist radioligand) | 14 | 84 | 88.7 | 86.3 |
| NK2 (h) (agonist radioligand) | 7 | 88.4 | 96.7 | 92.6 |
| Y1 (h) (agonist radioligand) | 1 | 99.8 | 98.3 | 99 |
| N neuronal alpha4beta2 (h) (agonist radioligand) | 3 | 91.1 | 103 | 97 |
| N muscle-type (h) (antagonist radioligand) | 1 | 95.2 | 103.6 | 99.4 |
| delta (DOP) (h) (agonist radioligand) | 76 | 24.4 | 24.1 | 24.2 |
| kappa (h) (KOP) (agonist radioligand) | 65 | 36.9 | 34 | 35.5 |
| mu (MOP) (h) (agonist radioligand) | 55 | 48.9 | 40.4 | 44.6 |
| NOP (ORL1) (h) (agonist radioligand) | 4 | 88.1 | 104.1 | 96.1 |
| PPARgamma (h) (agonist radioligand) | 32 | 70.7 | 64.3 | 67.5 |
| PCP (antagonist radioligand) | −18 | 132.9 | 103.2 | 118 |
| PAF (h) (agonist radioligand) | 7 | 91.2 | 95.7 | 93.4 |
| EP2 (h) (agonist radioligand) | −16 | 115.3 | 115.8 | 115.6 |
| FP (h) (agonist radioligand) | −24 | 126.6 | 121.6 | 124.1 |
| IP (PGI2) (h) (agonist radioligand) | 12 | 89.2 | 86.6 | 87.9 |
| LXR beta (h) agonist radioligand | 5 | 97 | 93.6 | 95.3 |
| RARalpha (h) (agonist radioligand) | −1 | 104.6 | 98.4 | 101.5 |
| 5-HT1A (h) (agonist radioligand) | −1 | 97.6 | 103.8 | 100.7 |
| 5-HT1B (h) (antagonist radioligand) | −8 | 114.1 | 101.7 | 107.9 |
| 5-HT1D (agonist radioligand) | 1 | 100.5 | 96.5 | 98.5 |
| 5-HT2A (h) (agonist radioligand) | 12 | 87.9 | 87.2 | 87.5 |
| 5-HT2B (h) (agonist radioligand) | 18 | 77.8 | 85.7 | 81.7 |
| 5-HT2C (h) (agonist radioligand) | 4 | 95.8 | 96.5 | 96.1 |
| 5-HT3 (h) (antagonist radioligand) | −8 | 107.4 | 109.2 | 108.3 |
| 5-HT4e (h) (antagonist radioligand) | 7 | 86.2 | 100.8 | 93.5 |
| 5-HT6 (h) (agonist radioligand) | −7 | 90.4 | 123.6 | 107 |
| 5-HT7 (h) (agonist radioligand) | −5 | 104.9 | 104.4 | 104.6 |
| sigma (non-selective) (h) (agonist radioligand) | 37 | 67.9 | 57.1 | 62.5 |
| sst1 (h) (agonist radioligand) | 0 | 91.4 | 107.8 | 99.6 |
| sst4 (h) (agonist radioligand) | 5 | 98 | 91.5 | 94.7 |
| GR (h) (agonist radioligand) | 55 | 49.2 | 41.5 | 45.3 |
| ER Alpha Human Estrogen NHR Binding | 8 | 89.9 | 94.7 | 92.3 |
| (Agonist radioligand) Assay, Cerep | | | | |
| AR(h) (agonist radioligand) | −7 | 96.1 | 117.3 | 106.7 |
| UT (h) (agonist radioligand) | 22 | 79.4 | 77 | 78.2 |
| VPAC1 (VIP1) (h) (agonist radioligand) | −9 | 102.7 | 115.1 | 108.9 |
| V1a (h) (agonist radioligand) | 15 | 84.7 | 84.6 | 84.7 |
| V2 (h) (agonist radioligand) | 9 | 87.2 | 94.6 | 90.9 |
| Ca2+ channel (L dihydropyridine site) | 2 | 101.9 | 94.1 | 98 |
| (antagonist radioligand) | | | | |
| Ca2+ channel (L verapamil site) | −6 | 109.6 | 102.3 | 105.9 |
| (phenylalkylamine) (antagonist radioligand) | | | | |
| Ca2+ channel (N) (antagonist radioligand) | −3 | 103.8 | 101.3 | 102.5 |
| SKCa channel (antagonist radioligand) | 2 | 97.6 | 97.6 | 97.6 |
| norepinephrine transporter (h) (antagonist | 8 | 92.1 | 91.9 | 92 |
| radioligand) | | | | |
| dopamine transporter (h) (antagonist radioligand) | −4 | 104.1 | 103.9 | 104 |
| GABA transporter (antagonist radioligand) | −1 | 100.7 | 101.4 | 101 |
| choline transporter (CHT1) (h) (antagonist | 9 | 92.5 | 90.3 | 91.4 |
| radioligand) | | | | |
| 5-HT transporter (h) (antagonist radioligand) | 1 | 103.2 | 95.8 | 99.5 |

Compound ID: 100074212-1 (WB3);
Test Concentration: 1.0E−05M

TABLE 4
Reference Compound Results

| | Reference | IC₅₀ | | |
|---|---|---|---|---|
| Assay | Compound | (M) | Kᵢ (M) | nH |
| Sodium Channel Site2 (Non-selective) Rat | Veratridine | 5.50E−06 | 2.10E−06 | 1.5 |
| Ion Channel Batrachotoxin Mass | | | | |
| Spectrometry Binding | | | | |
| Glutamate (Non-Selective) Rat Ion Channel | 5,7 dichloro- | 7.60E−08 | 3.50E−08 | 1 |
| Glycine (Strychnine-Insensitive) Mass | kynurenic acid | | | |
| Spectrometry Binding | | | | |
| Cav1.2 (L-type) Rat Calcium Ion Channel | Methoxy- | 8.60E−09 | 2.80E−09 | 0.7 |
| (Diltiazem Site) Mass Spectrometry Binding | verapamil (D600) | | | |
| GABAB(B1b/B2) Human GABA B GPCR | CGP 52432 | 6.50E−08 | 5.40E−08 | 0.8 |
| Mass Spectrometry Binding (Antagonist | | | | |
| Ligand) Assay, Cerep | | | | |
| A1 (h) (agonist radioligand) | CPA | 3.40E−09 | 1.40E−09 | 1.8 |
| A2A (h) (agonist radioligand) | NECA | 2.60E−08 | 2.20E−08 | 0.8 |
| A2B (h) (antagonist radioligand) | NECA | 7.10E−07 | 6.60E−07 | 0.7 |
| A3 (h) (agonist radioligand) | IB-MECA | 5.60E−10 | 3.30E−10 | 1.1 |
| alpha1A (h) (antagonist radioligand) | WB 4101 | 5.80E−10 | 2.90E−10 | 1.5 |
| alpha1B (h) (antagonist radioligand) | prazosin | 1.30E−10 | 3.50E−11 | 0.8 |
| alpha2A (h) (antagonist radioligand) | yohimbine | 5.30E−09 | 2.40E−09 | 1.2 |
| alpha2B (h) (antagonist radioligand) | yohimbine | 1.10E−08 | 7.20E−09 | 1.2 |
| alpha2C (h) (antagonist radioligand) | yohimbine | 3.30E−09 | 1.10E−09 | 0.7 |
| beta1 (h) (agonist radioligand) | atenolol | 2.70E−07 | 1.50E−07 | 1.3 |
| beta2 (h) (antagonist radioligand) | ICI 118551 | 7.50E−10 | 2.50E−10 | 1.3 |
| Beta3(h) (antagonist radioligand) | Alprenolol | 2.60E−07 | 2.20E−07 | 0.7 |
| AT1 (h) (antagonist radioligand) | saralasin | 2.60E−10 | 1.30E−10 | 0.5 |
| AT2 (h) (agonist radioligand) | angiotensin-II | 7.80E−11 | 3.90E−11 | 1.5 |
| APJ (apelin) (h) (agonist radioligand) | apelin-13, TFA | 1.40E−10 | 1.30E−10 | 0.8 |
| BZD (central) (agonist radioligand) | diazepam | 1.10E−08 | 9.70E−09 | 1.4 |
| BB3 (h) (agonist radioligand) | bombesin (6-14) | 3.40E−10 | 2.10E−10 | 0.7 |
| B2 (h) (agonist radioligand) | NPC 567 | 3.00E−08 | 1.50E−08 | 1.3 |
| CB2 (h) (agonist radioligand) | WIN 55212-2 | 9.30E−10 | 6.10E−10 | 0.9 |
| CB1 (h) (agonist radioligand) | CP 55940 | 6.70E−09 | 2.10E−09 | 1.4 |
| CCK1 (CCKA) (h) (agonist radioligand) | CCK-8s | 1.20E−10 | 9.10E−11 | 0.9 |
| CCK2 (CCKB) (h) (agonist radioligand) | CCK-8s | 8.30E−11 | 3.40E−11 | 0.8 |
| CRF1 (h) (agonist radioligand) | sauvagine | 4.00E−10 | 2.50E−10 | 1.1 |
| D1 (h) (antagonist radioligand) | SCH 23390 | 5.20E−10 | 2.10E−10 | 1.2 |
| D2S (h) (agonist radioligand) | 7-OH-DPAT | 4.20E−09 | 1.70E−09 | 1 |
| D3 (h) (antagonist radioligand) | (+)butaclamol | 2.20E−09 | 1.10E−09 | 1.1 |
| ETA (h) (agonist radioligand) | endothelin-1 | 5.40E−11 | 2.70E−11 | 0.8 |
| ETB (h) (agonist radioligand) | endothelin-3 | 2.60E−11 | 1.50E−11 | 1.2 |
| GABAA1 (h) (alpha1, beta2, gamma2) | muscimol | 7.00E−08 | 4.70E−08 | 1.1 |
| (agonist radioligand) | | | | |
| GABAA (Non-Selective) Rat Ion Channel | TBPS | 2.30E−08 | 1.60E−08 | 0.9 |
| Picrotoxinin Mass Spectrometry Binding | | | | |
| Antagonist Ligand | | | | |
| glucagon (h) (agonist radioligand) | glucagon | 6.70E−10 | 4.90E−10 | 0.8 |
| AMPA (agonist radioligand) | L-glutamate | 2.00E−07 | 1.80E−07 | 0.8 |
| kainate (agonist radioligand) | kainic acid | 4.20E−08 | 3.30E−08 | 0.9 |
| NMDA (antagonist radioligand) | CGS 19755 | 3.60E−07 | 3.00E−07 | 0.7 |
| mGluR1 (agonist radioligand) | L-Quisqualate | 9.80E−08 | 8.40E−08 | 1 |
| mGluR5 (h) (agonist radioligand) | L-Quisqualate | 7.10E−08 | 3.70E−08 | 0.9 |
| TNF-alpha (h) (agonist radioligand) | TNF-alpha | 2.70E−10 | 8.90E−11 | 1.1 |
| CCR2 (h) (agonist radioligand) | MCP-1 | 3.10E−11 | 1.30E−11 | 0.8 |
| H1 (h) (antagonist radioligand) | pyrilamine | 2.80E−09 | 1.70E−09 | 1 |
| H2 (h) (antagonist radioligand) | cimetidine | 6.90E−07 | 6.70E−07 | 0.9 |
| H3 (h) (agonist radioligand) | (R)alpha - | 7.60E−09 | 1.80E−09 | 1.9 |
| | Me-histamine | | | |
| H4 (h) (agonist radioligand) | imetit | 4.10E−09 | 1.80E−09 | 0.8 |
| CysLT1 (LTD4) (h) (agonist radioligand) | LTD4 | 3.40E−10 | 1.50E−10 | 1 |
| MCH1 (h) (agonist radioligand) | human MCH | 1.10E−10 | 1.00E−10 | 1.6 |
| MC1 (agonist radioligand) | NDP-alpha - MSH | 1.30E−10 | 6.60E−11 | 2 |
| MC3 (h) (agonist radioligand) | NDP-alpha - MSH | 2.00E−10 | 1.70E−10 | 1.4 |
| MC4 (h) (agonist radioligand) | NDP-alpha - MSH | 4.40E−10 | 4.00E−10 | 0.8 |
| MT1 (ML1A) (h) (agonist radioligand) | melatonin | 1.10E−10 | 8.80E−11 | 1 |
| MT3 (ML2) (agonist radioligand) | melatonin | 2.00E−07 | 2.00E−07 | 0.8 |
| MAO-A (antagonist radioligand) | clorgyline | 1.60E−09 | 9.30E−10 | 1.7 |
| motilin (h) (agonist radioligand) | [Nleu13]-motilin | 5.70E−10 | 4.80E−10 | 1.1 |
| M1 (h) (antagonist radioligand) | pirenzepine | 4.90E−08 | 4.30E−08 | 0.9 |
| M2 (h) (antagonist radioligand) | methoctramine | 4.90E−08 | 3.40E−08 | 1.1 |
| M3 (h) (antagonist radioligand) | 4-DAMP | 1.70E−09 | 1.20E−09 | 1.7 |
| M4 (h) (antagonist radioligand) | 4-DAMP | 8.10E−10 | 5.00E−10 | 1.1 |
| NK1 (h) (agonist radioligand) | [Sar9, | 3.40E−10 | 1.50E−10 | 1.1 |
| | Met(O2)11]-SP | | | |
| NK2 (h) (agonist radioligand) | [Nleu10]- | 3.50E−09 | 1.90E−09 | 0.8 |
| | NKA (4-10) | | | |
| Y1 (h) (agonist radioligand) | NPY | 2.90E−10 | 2.00E−10 | 0.7 |
| N neuronal alpha4beta2 (h) (agonist | nicotine | 6.80E−09 | 2.30E−09 | 1.1 |
| radioligand) | | | | |
| N muscle-type (h) (antagonist radioligand) | alpha - | 2.40E−09 | 1.90E−09 | 1.1 |
| | bungarotoxin | | | |
| delta (DOP) (h) (agonist radioligand) | DPDPE | 3.30E−09 | 1.80E−09 | 0.9 |
| kappa (h) (KOP) (agonist radioligand) | U50488 | 7.50E−10 | 4.10E−10 | 1.4 |
| mu (MOP) (h) (agonist radioligand) | DAMGO | 8.10E−10 | 3.30E−10 | 1.5 |
| NOP (ORL1) (h) (agonist radioligand) | nociceptin | 3.40E−10 | 1.20E−10 | 1 |
| PPARgamma (h) (agonist radioligand) | rosiglitazone | 1.40E−08 | 7.30E−09 | 1.3 |
| PCP (antagonist radioligand) | MK 801 | 1.10E−08 | 6.00E−09 | 1.1 |
| PAF (h) (agonist radioligand) | C16-PAF | 1.50E−09 | 9.10E−10 | 0.8 |
| EP2 (h) (agonist radioligand) | PGE2 | 4.40E−09 | 2.20E−09 | 1.2 |
| FP (h) (agonist radioligand) | PGF2alpha | 2.70E−09 | 1.80E−09 | 3 |
| IP (PGI2) (h) (agonist radioligand) | iloprost | 8.70E−09 | 5.00E−09 | 1 |
| LXR beta (h) agonist radioligand | T0901317 | 2.60E−08 | 1.50E−08 | 0.7 |
| RARalpha (h) (agonist radioligand) | all-trans- | 9.10E−09 | 3.10E−09 | 1.3 |
| | retinoic acid | | | |
| 5-HT1A (h) (agonist radioligand) | 8-OH-DPAT | 1.80E−09 | 9.20E−10 | 1.4 |
| 5-HT1B (h) (antagonist radioligand) | Serotonine | 2.20E−07 | 9.70E−08 | 0.9 |
| 5-HT1D (agonist radioligand) | serotonin | 1.60E−09 | 5.40E−10 | 1.1 |
| 5-HT2A (h) (agonist radioligand) | (±)DOI | 2.20E−10 | 1.70E−10 | 0.7 |
| 5-HT2B (h) (agonist radioligand) | (±)DOI | 3.40E−09 | 1.70E−09 | 0.8 |
| 5-HT2C (h) (agonist radioligand) | (±)DOI | 4.40E−10 | 4.00E−10 | 1 |
| 5-HT3 (h) (antagonist radioligand) | MDL 72222 | 1.10E−08 | 8.00E−09 | 1 |
| 5-HT4e (h) (antagonist radioligand) | serotonin | 1.70E−07 | 5.60E−08 | 0.8 |
| 5-HT6 (h) (agonist radioligand) | serotonin | 5.80E−08 | 2.70E−08 | 0.9 |
| 5-HT7 (h) (agonist radioligand) | serotonin | 4.40E−10 | 1.60E−10 | 1.6 |
| sigma (non-selective) (h) (agonist | haloperidol | 1.20E−07 | 9.90E−08 | 0.6 |
| radioligand) | | | | |
| sst1 (h) (agonist radioligand) | somatostatin-28 | 1.20E−09 | 1.10E−09 | 0.4 |
| sst4 (h) (agonist radioligand) | somatostatin-14 | 1.10E−09 | 1.10E−09 | 0.8 |
| GR (h) (agonist radioligand) | dexamethasone | 3.80E−09 | 1.90E−09 | 0.8 |
| ER Alpha Human Estrogen NHR Binding | 17 beta | 2.30E−10 | 9.10E−11 | 1 |
| (Agonist radioligand) Assay, Cerep | estradiol | | | |
| AR(h) (agonist radioligand) | testosterone | 8.50E−09 | 3.20E−09 | 1.3 |
| UT (h) (agonist radioligand) | urotensin-II | 1.20E−09 | 8.70E−10 | 0.9 |
| VPAC1 (VIP1) (h) (agonist radioligand) | VIP | 9.10E−11 | 5.00E−11 | 0.6 |
| V1a (h) (agonist radioligand) | [d(CH2)51, | 1.20E−09 | 7.30E−10 | 1.2 |
| | Tyr(Me)2]-AVP | | | |
| V2 (h) (agonist radioligand) | AVP | 6.80E−10 | 4.90E−10 | 0.9 |
| Ca2+ channel (L dihydropyridine site) | nitrendipine | 4.30E−10 | 2.20E−10 | 1.3 |
| (antagonist radioligand) | | | | |
| Ca2+ channel (L verapamil site) | D 600 | 8.60E−09 | 4.30E−09 | 0.7 |
| (phenylalkylamine) (antagonist radioligand) | | | | |
| Ca2+ channel (N) (antagonist radioligand) | omega - | 1.70E−12 | 6.90E−13 | 1.2 |
| | conotoxin GVIA | | | |
| SKCa channel (antagonist radioligand) | apamin | 2.80E−11 | 1.40E−11 | 1.3 |
| norepinephrine transporter (h) (antagonist | protriptyline | 4.70E−09 | 3.50E−09 | 1.2 |
| radioligand) | | | | |
| dopamine transporter (h) (antagonist radioligand) | BTCP | 9.10E−09 | 4.80E−09 | 0.7 |
| GABA transporter (antagonist radioligand) | nipecotic acid | 1.80E−06 | 1.80E−06 | 0.9 |
| choline transporter (CHT1) (h) (antagonist | hemicholinium-3 | 1.10E−08 | 6.50E−09 | 1 |
| radioligand) | | | | |
| 5-HT transporter (h) (antagonist radioligand) | imipramine | 4.70E−09 | 2.20E−09 | 1.1 |

### In Vitro Pharmacology: Enzyme and Uptake Assays

TABLE 5
Test Compound Results

| | % Inhibition | | | |
|---|---|---|---|---|
| | of Control | % of Control Values | | |
| Assay | Values | 1st | 2nd | Mean |
| Abl (ABL1) Human TK Kinase Enzymatic | 6 | 90 | 97.7 | 93.8 |
| Radiometric [Km ATP] | | | | |
| CaMK2alpha (CAMK2A) (Active) Human | −4 | 108.6 | 98.5 | 103.5 |
| CAMK Kinase Enzymatic Radiometric [Km ATP] | | | | |
| CDK2/CyclinA Human CMGC Kinase | 91 | 1 | 16.4 | 8.7 |
| Enzymatic Radiometric [Km ATP] | | | | |
| ERK2 (MAPK1) Human CMGC Kinase | 1 | 105.5 | 93.2 | 99.4 |
| Enzymatic Radiometric [Km ATP] | | | | |
| Flt-1 (VEGFR1) (FLT1) Human RTK Kinase | 11 | 91.3 | 86.1 | 88.7 |
| Enzymatic Radiometric [Km ATP] | | | | |
| Fyn Human TK Kinase Enzymatic | −4 | 104.4 | 103.4 | 103.9 |
| Radiometric [Km ATP] | | | | |
| LYN Human TK Kinase Enzymatic | −17 | 115.2 | 118.3 | 116.7 |
| Radiometric [Km ATP] | | | | |
| p38alpha (SAPK2A) Human CMGC Kinase | −1 | 102.4 | 98.7 | 100.5 |
| Enzymatic Radiometric [Km ATP] | | | | |
| ZAP70 Human TK Kinase Enzymatic | 16 | 91.2 | 76.2 | 83.7 |
| Radiometric Assay [Km ATP] | | | | |
| COX1(h) | −18 | 125.6 | 110 | 117.8 |
| COX2(h) | −10 | 116.7 | 103.1 | 109.9 |
| inducible NOS | 3 | 98.4 | 95.3 | 96.9 |
| PDE2A1 (h) | −29 | 111.6 | 145.5 | 128.5 |
| PDE3B (h) | 17 | 78.3 | 87.7 | 83 |
| PDE4D2 (h) | 5 | 95.8 | 94.2 | 95 |
| PDE5 (h) (non-selective) | −5 | 102.1 | 108.8 | 105.5 |
| PDE6 (non-selective) | 6 | 90.2 | 98.4 | 94.3 |
| ACE-2 (h) | −17 | 114.3 | 119.9 | 117.1 |
| ACE (h) | −9 | 108.3 | 109.5 | 108.9 |
| BACE-1 (h) (beta-secretase) | 10 | 91.6 | 89.2 | 90.4 |
| caspase-3 (h) | 1 | 98.7 | 98.9 | 98.8 |
| HIV-1 protease | 1 | 101 | 96.4 | 98.7 |
| MMP-1 (h) | 4 | 95.3 | 95.9 | 95.6 |
| IR (INSR) Human RTK Kinase Enzymatic | 9 | 80.2 | 101.4 | 90.8 |
| Radiometric [Km ATP] | | | | |
| acetylcholinesterase (h) | 7 | 93.1 | 93.8 | 93.5 |
| xanthine oxidase/superoxide O2-scavenging | −5 | 106.5 | 103.5 | 105 |
| ATPase (Na+/K+) | 2 | 92.8 | 103.7 | 98.2 |

Compound ID: 100074212-1 (WB3);
Test Concentration: 1.0E−05M

TABLE 6
Reference Compound Results

| | Reference | | nH |
|---|---|---|---|
| Assay | Compound | IC₅₀ (M) | Ref |
| Abl (ABL1) Human TK Kinase Enzymatic | Staurosporine | 2.60E−07 | 0.9 |
| Radiometric [Km ATP] | | | |
| CaMK2alpha (CAMK2A) (Active) Human | Staurosporine | 1.60E−10 | 1.4 |
| CAMK Kinase Enzymatic Radiometric | | | |
| [Km ATP] | | | |
| CDK2/CyclinA Human CMGC Kinase | Staurosporine | 8.90E−09 | 1.1 |
| Enzymatic Radiometric [Km ATP] | | | |
| ERK2 (MAPK1) Human CMGC Kinase | PKR Inhibitor | 9.60E−08 | 1.1 |
| Enzymatic Radiometric [Km ATP] | | | |
| Flt-1 (VEGFR1) (FLT1) Human RTK | Staurosporine | 3.40E−09 | 1.1 |
| Kinase Enzymatic Radiometric [Km ATP] | | | |
| Fyn Human TK Kinase Enzymatic | Staurosporine | 4.10E−09 | 1.1 |
| Radiometric [Km ATP] | | | |
| LYN Human TK Kinase Enzymatic | Staurosporine | 1.50E−09 | 1.1 |
| Radiometric [Km ATP] | | | |
| p38alpha (SAPK2A) Human CMGC | 1-NM-PP1 | 2.30E−06 | 1 |
| Kinase Enzymatic Radiometric [Km ATP] | | | |
| ZAP70 Human TK Kinase Enzymatic | Staurosporine | 6.50E−08 | 1.3 |
| Radiometric Assay [Km ATP] | | | |
| COX1(h) | Diclofenac | 1.40E−08 | 1.9 |
| COX2(h) | NS398 | 1.90E−07 | 1.1 |
| inducible NOS | 1400W | 3.20E−08 | 1.1 |
| PDE2A1 (h) | BAY60-7550 | 2.70E−09 | 1.5 |
| PDE3B (h) | milrinone | 2.50E−06 | 1 |
| PDE4D2 (h) | Ro 20-1724 | 4.60E−07 | 0.7 |
| PDE5 (h) (non-selective) | dipyridamole | 1.50E−06 | 0.9 |
| PDE6 (non-selective) | zaprinast | 1.80E−07 | 0.9 |
| ACE-2 (h) | Ac-GG-26-NH2 | 1.00E−06 | 2.5 |
| ACE (h) | captopril | 7.90E−10 | 1.3 |
| BACE-1 (h) (beta-secretase) | OM 99-2 | 1.80E−07 | 1.8 |
| caspase-3 (h) | Ac-DEVD-CHO | 3.20E−09 | 1.5 |
| HIV-1 protease | pepstatin A | 5.10E−07 | 1.6 |
| MMP-1 (h) | GM6001 | 1.50E−09 | 1.5 |
| IR (INSR) Human RTK Kinase Enzymatic | Staurosporine | 1.90E−07 | 0.9 |
| Radiometric [Km ATP] | | | |
| acetylcholinesterase (h) | galanthamine | 5.10E−07 | 1 |
| xanthine oxidase/superoxide O2-scavenging | allopurinol | 2.30E−06 | 1.4 |
| ATPase (Na+/K+) | ouabain | 3.20E−07 | 0.9 |

TABLE 7
Test Compound Results

| | | % Stimulation | | |
|---|---|---|---|---|
| | % Stimulation | Relative to Control | | |
| Assay | Relative to Control | 1st | 2nd | Mean |
| guanylyl cyclase (h) | 1 | 2.5 | 0.2 | 1.3 |
| (activator effect) | | | | |

Compound ID: 100074212-1 (WB3);
Test Concentration: 1.0E−05M

TABLE 8
Reference Compound Results

| Assay | Reference Compound | EC₅₀ (M) | nH |
|---|---|---|---|
| guanylyl cyclase (h) | sodium nitroprusside | 5.4E−06 | 0.7 |
| (activator effect) | | | |

### In Vitro Pharmacology

Results showing an inhibition (or stimulation for assays run in basal conditions) higher than 50% are considered to represent significant effects of the test compounds. 50% is the most common cut-off value for further investigation (determination of IC₅₀ or EC₅₀ values from concentration-response curves) that we would recommend.

Results showing an inhibition (or stimulation) between 25% and 50% are indicative of weak to moderate effects (in most assays, they should be confirmed by further testing as they are within a range where more inter-experimental variability can occur).

Results showing an inhibition (or stimulation) lower than 25% are not considered significant and mostly attributable to variability of the signal around the control level.

Low to moderate negative values have no real meaning and are attributable to variability of the signal around the control level. High negative values (≥50%) that are sometimes obtained with high concentrations of test compounds are generally attributable to non-specific effects of the test compounds in the assays.

### 9. Materials and Methods

### 9.1. Experimental Conditions

Minor variations to the experimental protocol described below may have occurred during the testing.

| | | | | | | | | |
|---|---|---|---|---|---|---|---|---|
| Assay | Source | Ligand | Conc. | Kd | Non Specific | Incubation | Detection | Bibl. |

Customized assays

| | | | | | | | | | | |
|---|---|---|---|---|---|---|---|---|---|---|
| GABAA (Non- | rat brain | Picrotoxinin | 20 | nM | 47 | nM | TBPS | 120 minutes 22° C. | MS | 1664 |
| Selective) Rat | | | | | | | (10 μM) | | | |
| Ion Channel | | | | | | | | | | |
| Picrotoxinin | | | | | | | | | | |
| Mass | | | | | | | | | | |
| Spectrometry | | | | | | | | | | |
| Binding | | | | | | | | | | |
| Antagonist | | | | | | | | | | |
| Ligand | | | | | | | | | | |

Receptors

| | | | | | | | | | | |
|---|---|---|---|---|---|---|---|---|---|---|
| GABAB(B1b/ | human | CGP 54626 | 1 | nM | 5 | nM | CGP | 120 min at RT | MS | 508 |
| B2) Human | recombinant | | | | | | 52432 | | | |
| GABA B | (CHO cells) | | | | | | (100 μM) | | | |
| GPCR Mass | | | | | | | | | | |
| Spectrometry | | | | | | | | | | |
| Binding | | | | | | | | | | |
| (Antagonist | | | | | | | | | | |
| Ligand) | | | | | | | | | | |
| Assay, Cerep | | | | | | | | | | |
| A₁ (h) | human | [³H]CCPA | 1 | nM | 0.7 | nM | CPA | 60 min RT | Scintillation | 198 |
| (agonist | recombinant | | | | | | (10 μM) | | counting | |
| radioligand) | (CHO cells) | | | | | | | | | |
| A_{2A} (h) | human | [³H]CGS | 6 | nM | 27 | nM | NECA | 120 min RT | Scintillation | 141 |
| (agonist | recombinant | 21680 | | | | | (10 μM) | | | |
| radioligand) | (HEK-293 | | | | | | | | counting | |
| | cells) | | | | | | | | | |
| A_{2B} (h) | human | [³H]CPX | 5 | nM | 65 | nM | NECA | 60 min RT | Scintillation | 229 |
| (antagonist | recombinant | | | | | | (100 μM) | | counting | |
| radioligand) | (HEK-293 | | | | | | | | | |
| | cells) | | | | | | | | | |
| A₃ (h) | human | [¹²⁵I]AB-MECA | 0.15 | nM | 0.22 | nM | IB-MECA | 120 min RT | Scintillation | 206 |
| (agonist | recombinant | | | | | | (1 μM) | | counting | |
| radioligand) | (HEK-293 | | | | | | | | | |
| | cells) | | | | | | | | | |
| alpha_{1A} (h) | human | [³H]prazosin | 0.1 | nM | 0.1 | nM | epinephrine | 60 min RT | Scintillation | 897 |
| (antagonist | recombinant | | | | | | (0.1 mM) | | counting | |
| radioligand) | (CHO cells) | | | | | | | | | |
| alpha_{1B} (h) | human | [³H]prazosin | 0.15 | nM | 0.055 | nM | phentolamine | 60 min RT | Scintillation | 701 |
| (antagonist | recombinant | | | | | | (10 μM) | | counting | |
| radioligand) | (CHO cells) | | | | | | | | | |
| alpha_{2A} (h) | human | [³H]RX 821002 | 1 | nM | 0.8 | nM | (−)epinephrine | 60 min RT | Scintillation | 542 |
| (antagonist | recombinant | | | | | | (100 μM) | | counting | |
| radioligand) | (CHO cells) | | | | | | | | | |
| alpha_{2B} (h) | human | [³H]RX 821002 | 2.5 | nM | 5 | nM | (−)epinephrine | 60 min RT | Scintillation | 56 |
| (antagonist | recombinant | | | | | | (100 μM) | | counting | |
| radioligand) | (CHO cells) | | | | | | | | | |
| alpha_{2C} (h) | human | [³H]RX 821002 | 2 | nM | 0.95 | nM | (−)epinephrine | 60 min RT | Scintillation | 56 |
| (antagonist | recombinant | | | | | | (100 μM) | | counting | |
| radioligand) | (CHO cells) | | | | | | | | | |

| | | | | | | | | | Detection | |
|---|---|---|---|---|---|---|---|---|---|---|
| Assay | Source | Ligand | Conc. | | Kd | | Non Specific | Incubation | Method | Bibl. |
| beta1 (h) | human | [3H](−)CGP | 0.3 | nM | 0.39 | nM | alprenolol | 60 min RT | Scintillation | 548 |
| (agonist | recombinant | 12177 | | | | | (50 μM) | | counting | |
| radioligand) | (HEK-293 | | | | | | | | | |
| | cells) | | | | | | | | | |
| beta2 (h) | human | [3H](−)CGP | 0.3 | nM | 0.15 | nM | alprenolol | 120 min RT | Scintillation | 794 |
| (antagonist | recombinant | 12177 | | | | | (50 μM) | | counting | |
| radioligand) | (CHO cells) | | | | | | | | | |
| Beta3(h) | human | [125I] | 0.5 | nM | 2.9 | nM | Alprenolol | 60 min RT | Scintillation | 1277 |
| (antagonist | recombinant | Cyanopindolol | | | | | (100 μM) | | counting | |
| radioligand) | (HEK-293 | | | | | | | | | |
| | cells) | | | | | | | | | |
| AT1 (h) | human | [125I][Sar1, | 0.05 | nM | 0.05 | nM | angiotensin-II | 120 min 37° C. | Scintillation | 776 |
| (antagonist | recombinant | Ile8]- | | | | | (10 μM) | | counting | |
| radioligand) | (HEK-293 | AT-II | | | | | | | | |
| | cells) | | | | | | | | | |
| AT2 (h) | human | [125I]CGP | 0.01 | nM | 0.01 | nM | angiotensin-II | 4 hr 37° C. | Scintillation | 248 |
| (agonist | recombinant | 42112A | | | | | (1 μM) | | counting | |
| radioligand) | (HEK-293 | | | | | | | | | |
| | cells) | | | | | | | | | |
| APJ (apelin) | human | [125I] | 0.03 | nM | 0.06 | nM | apelin-13 | 120 min RT | Scintillation | 846 |
| (h) | recombinant | (Glp65, Nle75, | | | | | (1 μM) | | counting | |
| (agonist | (CHO cells) | Tyr77)-apelin-13 | | | | | | | | |
| radioligand) | | | | | | | | | | |
| BB3 (h) | human | [125I]Bn(6-14) | 0.1 | nM | 0.16 | nM | Bn (6-14) | 60 min RT | Scintillation | 287 |
| (agonist | recombinant | | | | | | (1 μM) | | counting | |
| radioligand) | (CHO cells) | | | | | | | | | |
| B2 (h) | human | [3H]bradykinin | 0.3 | nM | 0.32 | nM | bradykinin | 60 min RT | Scintillation | 346 |
| (agonist | recombinant | | | | | | (1 μM) | | counting | |
| radioligand) | (CHO cells) | | | | | | | | | |
| CB2 (h) | human | [3H]WIN | 0.8 | nM | 1.5 | nM | WIN 55212-2 | 120 min 37° C. | Scintillation | 165 |
| (agonist | recombinant | 55212-2 | | | | | (5 μM) | | counting | |
| radioligand) | (CHO cells) | | | | | | | | | |
| CB1 (h) | human | [3H]CP 55940 | 2 | nM | 0.9 | nM | AM281 | 30 min 22° C. | Scintillation | 657 |
| (agonist | recombinant | | | | | | (10 μM) | | counting | |
| radioligand) | (Chem-RBL | | | | | | | | | |
| | cells) | | | | | | | | | |
| CCK1 (CCKA) | human | [125I]CCK-8s | 0.08 | nM | 0.24 | nM | CCK-8s | 60 min RT | Scintillation | 562 |
| (h) | recombinant | | | | | | (1 μM) | | counting | |
| (agonist | (CHO cells) | | | | | | | | | |
| radioligand) | | | | | | | | | | |
| CCK2 (CCKB) | human | [125I]CCK-8s | 0.08 | nM | 0.054 | nM | CCK-8s | 60 min RT | Scintillation | 134 |
| (h) | recombinant | | | | | | (1 μM) | | counting | |
| (agonist | (CHO cells) | | | | | | | | | |
| radioligand) | | | | | | | | | | |
| CRF1 (h) | human | [125I]sauvagine | 0.075 | nM | 0.12 | nM | sauvagine | 120 min RT | Scintillation | 557 |
| (agonist | recombinant | | | | | | (0.5 μM) | | counting | |
| radioligand) | (CHO cells) | | | | | | | | | |
| D1 (h) | human | [3H]SCH 23390 | 0.3 | nM | 0.2 | nM | SCH 23390 | 60 min RT | Scintillation | 281 |
| (antagonist | recombinant | | | | | | (1 μM) | | counting | |
| radioligand) | (CHO cells) | | | | | | | | | |
| D2S (h) | human | [3H]7-OH- | 1 | nM | 0.68 | nM | butaclamol | 60 min RT | Scintillation | 87 |
| (agonist | recombinant | DPAT | | | | | (10 μM) | | counting | |
| radioligand) | (HEK-293 | | | | | | | | | |
| | cells) | | | | | | | | | |
| D3 (h) | human | [3H]methyl- | 0.25 | nM | 0.25 | nM | (+)butaclamol | 60 min RT | Scintillation | 145 |
| (antagonist | recombinant | spiperone | | | | | (10 μM) | | counting | |
| radioligand) | (CHO cells) | | | | | | | | | |
| ETA (h) | human | [125I]endothelin-1 | 0.03 | nM | 0.03 | nM | endothelin-1 | 120 min 37° C. | Scintillation | 30 |
| (agonist | recombinant | | | | | | (100 nM) | | counting | |
| radioligand) | (CHO cells) | | | | | | | | | |
| ETB (h) | human | [125I]endothelin-1 | 0.03 | nM | 0.04 | nM | endothelin-1 | 120 min 37° C. | Scintillation | 541 |
| (agonist | recombinant | | | | | | (0.1 μM) | | counting | |
| radioligand) | (CHO cells) | | | | | | | | | |
| GABAA1 (h) | human | [3H]muscimol | 15 | nM | 30 | nM | muscimol | 120 min RT | Scintillation | 1096 |
| (alpha1, beta2, | recombinant | | | | | | (10 μM) | | counting | |
| gamma2) | (CHO cells) | | | | | | | | | |
| (agonist | | | | | | | | | | |
| radioligand) | | | | | | | | | | |
| glucagon (h) | human | [125I]glucagon | 0.025 | nM | 0.069 | nM | glucagon | 120 min RT | Scintillation | 624 |
| (agonist | recombinant | | | | | | (1 μM) | | counting | |
| radioligand) | (CHO cells) | | | | | | | | | |
| mGluR1 | rat cerebellum | [3H]quisqualate | 40 | nM | 240 | nM | L-Glutamate | 60 min RT | Scintillation | 1222 |
| (agonist | | | | | | | (1 mM) | | counting | |
| radioligand) | | | | | | | | | | |
| mGluR5 (h) | human | [3H]Quisqualate | 40 | nM | 44 | nM | L-Glutamate | 120 min RT | Scintillation | 1222 |
| (agonist | recombinant | | | | | | (1 mM) | | counting | |
| radioligand) | (CHO cells) | | | | | | | | | |
| TNF-alpha (h) | human | [125I]TNF-α | 0.1 | nM | 0.05 | nM | TNF-α | 120 min 4° C. | Scintillation | 26 |
| (agonist | endogenous | | | | | | (10 nM) | | counting | |
| radioligand) | (U-937 cells) | | | | | | | | | |
| CCR2 (h) | human | [125I]MCP-1 | 0.01 | nM | 0.007 | nM | MCP-1 | 60 min RT | Scintillation | 13 |
| (agonist | recombinant | | | | | | (10 nM) | | counting | |
| radioligand) | (HEK-293 | | | | | | | | | |
| | cells) | | | | | | | | | |
| H1 (h) | human | [3H]pyrilamine | 1 | nM | 1.7 | nM | pyrilamine | 60 min RT | Scintillation | 492 |
| (antagonist | recombinant | | | | | | (1 μM) | | counting | |
| radioligand) | (HEK-293 | | | | | | | | | |
| | cells) | | | | | | | | | |
| H2 (h) | human | [125I]APT | 0.075 | nM | 2.9 | nM | tiotidine | 120 min RT | Scintillation | 540 |
| (antagonist | recombinant | | | | | | (100 μM) | | counting | |
| radioligand) | (CHO cells) | | | | | | | | | |
| H3 (h) | human | [3H]Nα-Me- | 1 | nM | 0.32 | nM | (R)α-Me- | 60 min RT | Scintillation | 563 |
| (agonist | recombinant | histamine | | | | | histamine | | counting | |
| radioligand) | (CHO cells) | | | | | | (1 μM) | | | |
| H4 (h) | human | [3H]histamine | 10 | nM | 7.6 | nM | imetit | 60 min RT | Scintillation | 631 |
| (agonist | recombinant | | | | | | (1 μM) | | counting | |
| radioligand) | (HEK-293 | | | | | | | | | |
| | cells) | | | | | | | | | |
| CysLT1 (LTD4) | human | [3H]LTD4 | 0.3 | nM | 0.24 | nM | LTD4 | 60 min RT | Scintillation | 618 |
| (h) | recombinant | | | | | | (1 μM) | | counting | |
| (agonist | (CHO cells) | | | | | | | | | |
| radioligand) | | | | | | | | | | |
| MCH1 (h) | human | [125I] | 0.1 | nM | 1 | nM | human MCH | 60 min RT | Scintillation | 526 |
| (agonist | recombinant | [Phe13, Tyr19]- | | | | | (0.1 μM) | | counting | |
| radioligand) | (CHO cells) | MCH | | | | | | | | |
| MC1 | mouse | [125I]NDP-α- | 0.05 | nM | 0.05 | nM | NDP-α-MSH | 90 min RT | Scintillation | 390 |
| (agonist | endogenous | MSH | | | | | (1 μM) | | counting | |
| radioligand) | (B16-F1 cells) | | | | | | | | | |
| MC3 (h) | human | [125I]NDP-α- | 0.075 | nM | 0.4 | nM | NDP-α-MSH | 60 min 37° C. | Scintillation | 211 |
| (agonist | recombinant | MSH | | | | | (1 μM) | | counting | |
| radioligand) | (CHO cells) | | | | | | | | | |
| MC4 (h) | human | [125I]NDP-α- | 0.05 | nM | 0.54 | nM | NDP-α-MSH | 120 min 37° C. | Scintillation | 211 |
| (agonist | recombinant | MSH | | | | | (1 μM) | | counting | |
| radioligand) | (CHO cells) | | | | | | | | | |
| MT1 (ML1A) | human | [125I]2- | 0.01 | nM | 0.04 | nM | melatonin | 240 min RT | Scintillation | 639 |
| (h) | recombinant | iodomelatonin | | | | | (1 μM) | | counting | |
| (agonist | (CHO cells) | | | | | | | | | |
| radioligand) | | | | | | | | | | |
| MT3 (ML2) | hamster brain | [125I]2- | 0.1 | nM | 4.8 | nM | melatonin | 60 min 4° C. | Scintillation | 186 |
| (agonist | | iodomelatonin | | | | | (30 μM) | | counting | |
| radioligand) | | | | | | | | | | |
| motilin (h) | human | [125I]motilin | 0.05 | nM | 0.26 | nM | [Nleu13]-motilin | 120 min RT | Scintillation | 285 |
| (agonist | recombinant | | | | | | (1 μM) | | counting | |
| radioligand) | (CHO cells) | | | | | | | | | |
| M1 (h) | human | [3H]pirenzepine | 2 | nM | 13 | nM | atropine | 60 min RT | Scintillation | 59 |
| (antagonist | recombinant | | | | | | (1 μM) | | counting | |
| radioligand) | (CHO cells) | | | | | | | | | |
| M2 (h) | human | [3H]AF-DX 384 | 2 | nM | 4.6 | nM | atropine | 60 min RT | Scintillation | 59 |
| (antagonist | recombinant | | | | | | (1 μM) | | counting | |
| radioligand) | (CHO cells) | | | | | | | | | |
| M3 (h) | human | [3H]4-DAMP | 0.2 | nM | 0.5 | nM | atropine | 60 min RT | Scintillation | 546 |
| (antagonist | recombinant | | | | | | (1 μM) | | counting | |
| radioligand) | (CHO cells) | | | | | | | | | |
| M4 (h) | human | [3H]4-DAMP | 0.2 | nM | 0.32 | nM | atropine | 60 min RT | Scintillation | 59 |
| (antagonist | recombinant | | | | | | (1 μM) | | counting | |
| radioligand) | (CHO cells) | | | | | | | | | |
| NK1 (h) | human | [125I]- | 0.05 | nM | 0.04 | nM | [Sar9, | 30 min RT | Scintillation | 104 |
| (agonist | endogenous | Substance | | | | | Met(O2)11]-SP | | counting | |
| radioligand) | (U373MG | PLYS3 | | | | | (1 μM) | | | |
| | cells) | | | | | | | | | |
| NK2 (h) | human | [125I]NKA | 0.1 | nM | 0.12 | nM | [Nleu10]-NKA | 60 min RT | Scintillation | 3 |
| (agonist | recombinant | | | | | | (4-10) | | counting | |
| radioligand) | (CHO cells) | | | | | | (300 nM) | | | |
| Y1 (h) | human | [125I]peptide | 0.025 | nM | 0.06 | nM | NPY | 120 min 37° C. | Scintillation | 391 |
| (agonist | endogenous | YY | | | | | (1 μM) | | counting | |
| radioligand) | (SK-N-MC | | | | | | | | | |
| | cells) | | | | | | | | | |
| N neuronal | human | [3H]cytisine | 0.6 | nM | 0.3 | nM | nicotine | 120 min 4° C. | Scintillation | 1084 |
| alpha4beta2 | recombinant | | | | | | (10 μM) | | counting | |
| (h) | cells) | | | | | | | | | |
| (agonist | (SH-SY5Y | | | | | | | | | |
| radioligand) | | | | | | | | | | |
| N muscle-type | human | [125I]α- | 0.5 | nM | 2 | nM | α-bungarotoxin | 120 min RT | Scintillation | 524 |
| (h) | endogenous | bungarotoxin | | | | | (5 μM) | | counting | |
| (antagonist | (TE671 cells) | | | | | | | | | |
| radioligand) | | | | | | | | | | |
| delta (DOP) | human | [3H]DADLE | 0.5 | nM | 0.6 | nM | naltrexone | 60 min RT | Scintillation | 501 |
| (h) (agonist | recombinant | | | | | | (10 μM) | | counting | |
| radioligand) | (Chem-1 (RBL) | | | | | | | | | |
| | cells) | | | | | | | | | |
| kappa (h) | human | [3H]U69593 | 0.5 | nM | 0.6 | nM | naloxone | 60 min RT | Scintillation | 222 |
| (KOP) | recombinant | | | | | | (10 μM) | | counting | |
| (agonist | (RBL cells) | | | | | | | | | |
| radioligand) | | | | | | | | | | |
| μ (MOP) (h) | human | [3H]DAMGO | 0.5 | nM | 0.35 | nM | naloxone | 120 min RT | Scintillation | 260 |
| (agonist | recombinant | | | | | | (10 μM) | | counting | |
| radioligand) | (HEK-293 | | | | | | | | | |
| | cells) | | | | | | | | | |
| NOP (ORL1) | human | [3H]nociceptin | 0.1 | nM | 0.054 | nM | nociceptin | 60 min RT | Scintillation | 1588 |
| (h) (agonist | recombinant | | | | | | (1 μM) | | counting | |
| radioligand) | (Chem-1 (RBL) | | | | | | | | | |
| | cells) | | | | | | | | | |
| PPARgamma | human | [3H]rosiglitazone | 5 | nM | 5.7 | nM | rosiglitazone | 120 min 4° C. | Scintillation | 567 |
| (h) | recombinant | | | | | | (10 μM) | | counting | |
| (agonist | (E. coli) | | | | | | | | | |
| radioligand) | | | | | | | | | | |
| PAF (h) | human | [3H]C18-PAF | 1 | nM | 1.5 | nM | C16-PAF | 180 min RT | Scintillation | 531 |
| (agonist | recombinant | | | | | | (10 μM) | | counting | |
| radioligand) | (CHO cells) | | | | | | | | | |
| EP2 (h) | human | [3H]PGE2 | 3 | nM | 3 | nM | PGE2 | 120 min RT | Scintillation | 781 |
| (agonist | recombinant | | | | | | (10 μM) | | counting | |
| radioligand) | (HEK-293 | | | | | | | | | |
| | cells) | | | | | | | | | |
| FP (h) | human | [3H]PGF2α | 2 | nM | 3.83 | nM | cloprostenol | 60 min RT | Scintillation | 781 |
| (agonist | recombinant | | | | | | (10 μM) | | counting | |
| radioligand) | (HEK-293 | | | | | | | | | |
| | cells) | | | | | | | | | |
| IP (PGI2) (h) | human | [3H]iloprost | 6 | nM | 8 | nM | iloprost | 60 min RT | Scintillation | 781 |
| (agonist | recombinant | | | | | | (10 μM) | | counting | |
| radioligand) | (HEK-293 | | | | | | | | | |
| | cells) | | | | | | | | | |
| LXR beta (h) | human | [3H] T0901317 | 20 | nM | 27 | nM | T0901317 | 240 min 4° C. | Scintillation | 1578 |
| agonist | recombinant | | | | | | (10 μM) | | counting | |
| radioligand | (E. coli) | | | | | | | | | |
| RARalpha (h) | human | 9-cis-Retinoic | 1 | nM | 0.52 | | all-trans | 120 min 4° C. | Scintillation | 855 |
| (agonist | recombinant | Acid | | | | | retinoic acid | | counting | |
| radioligand) | (insect cells) | [11, 12-3H] | | | | | (1 μM) | | | |
| 5-HT1A (h) | human | [3H]8-OH- | 0.5 | nM | 0.5 | nM | 8-OH-DPAT | 60 min RT | Scintillation | 164 |
| (agonist | recombinant | DPAT | | | | | (10 μM) | | counting | |
| radioligand) | (HEK-293 | | | | | | | | | |
| | cells) | | | | | | | | | |
| 5-HT1B (h) | human | [3H]GR125743 | 1 | nM | 0.8 | nM | Serotonine | 60 min 37° C. | Scintillation | 1451 |
| (antagonist | recombinant | | | | | | (30 μM) | | counting | |
| radioligand) | (Chem-1 (RBL) | | | | | | | | | |
| | cells) | | | | | | | | | |
| 5-HT1D | rat recombinant | [3H]serotonin | 1 | nM | 0.5 | nM | serotonin | 60 min RT | Scintillation | 777 |
| (agonist | (CHO cells) | | | | | | (10 μM) | | counting | |
| radioligand) | | | | | | | | | | |
| 5-HT2A (h) | human | [125I](±)DOI | 0.1 | nM | 0.3 | nM | (±)DOI | 60 min RT | Scintillation | 288 |
| (agonist | recombinant | | | | | | (1 μM) | | counting | |
| radioligand) | (HEK-293 | | | | | | | | | |
| | cells) | | | | | | | | | |
| 5-HT2B (h) | human | [125I](±)DOI | 0.2 | nM | 0.2 | nM | (±)DOI | 60 min RT | Scintillation | 571 |
| (agonist | recombinant | | | | | | (1 M) | | counting | |
| radioligand) | (CHO cells) | | | | | | | | | |
| 5-HT2C (h) | human | [125I](±)DOI | 0.1 | nM | 0.9 | nM | (±)DOI | 60 min 37° C. | Scintillation | 288 |
| (agonist | recombinant | | | | | | (10 μM) | | counting | |
| radioligand) | (HEK-293 | | | | | | | | | |
| | cells) | | | | | | | | | |
| 5-HT4e (h) | human | [3H]GR 113808 | 0.3 | nM | 0.15 | nM | serotonin | 60 min 37° C. | Scintillation | 309 |
| (antagonist | recombinant | | | | | | (100 μM) | | counting | |
| radioligand) | (CHO cells) | | | | | | | | | |
| 5-HT6 (h) | human | [3H]LSD | 2 | nM | 1.8 | nM | serotonin | 120 min 37° C. | Scintillation | 161 |
| (agonist | recombinant | | | | | | (100 μM) | | counting | |
| radioligand) | (CHO cells) | | | | | | | | | |
| 5-HT7 (h) | human | [3H]LSD | 4 | nM | 2.3 | nM | serotonin | 120 min RT | Scintillation | 217 |
| (agonist | recombinant | | | | | | (10 μM) | | counting | |
| radioligand) | (CHO cells) | | | | | | | | | |
| sigma (non- | human | [3H]DTG | 10 | nM | 41 | nM | Haloperidol | 120 min RT | Scintillation | 1136 |
| selective) (h) | endogenous | | | | | | (10 μM) | | counting | |
| (agonist | (Jurkat cells) | | | | | | | | | |
| radioligand) | | | | | | | | | | |
| sst1 (h) | human | [125I]Tyr11- | 0.1 | nM | 1 | nM | somatostatin-28 | 180 min 37° C. | Scintillation | 761 |
| (agonist | recombinant | somatostatin-14 | | | | | (1 μM) | | counting | |
| radioligand) | (CHO cells) | | | | | | | | | |
| sst4 (h) | human | [125I]Tyr11- | 0.1 | nM | 5.9 | nM | somatostatin-14 | 120 min RT | Scintillation | 296 |
| (agonist | recombinant | somatostatin-14 | | | | | (1 μM) | | counting | |
| radioligand) | (CHO cells) | | | | | | | | | |
| GR (h) | human | [3H]dexamethasone | 1.5 | nM | 1.5 | nM | triamcinolone | 24 hr 4° C. | Scintillation | 283 |
| (agonist | endogenous | | | | | | (10 μM) | | counting | |
| radioligand) | (IM-9 cells) | | | | | | | | | |
| ER Alpha | human | [3H] Estradiol | 0.4 | nM | 0.26 | nM | Diethylstilbestol | 120 min 22° C. | Scintillation | 1280 |
| Human | recombinant | | | | | | (1 μM) | | counting | |
| Estrogen NHR | | | | | | | | | | |
| Binding | | | | | | | | | | |
| (Agonist | | | | | | | | | | |
| radioligand) | | | | | | | | | | |
| Assay, Cerep | | | | | | | | | | |
| AR(h) | human | [3H]methyltrienolone | 1 | nM | 0.6 | nM | testosterone | 4 hr 22° C. | Scintillation | 498 |
| (agonist | endogenous | | | | | | (1 M) | | counting | |
| radioligand) | (LNCaP cells) | | | | | | | | | |
| UT (h) | human | [125I]urotensin- | 0.1 | nM | 0.29 | nM | urotensin-II | 120 min RT | Scintillation | 622 |
| (agonist | recombinant | II | | | | | (3 μM) | | counting | |
| radioligand) | (CHO cells) | | | | | | | | | |
| VPAC1 (VIP1) | human | [125I]VIP | 0.04 | nM | 0.05 | nM | VIP | 60 min RT | Scintillation | 50 |
| (h) | recombinant | | | | | | (1 μM) | | counting | |
| (agonist | (CHO cells) | | | | | | | | | |
| radioligand) | | | | | | | | | | |
| V1a (h) | human | [3H]AVP | 0.3 | nM | 0.5 | nM | AVP | 60 min RT | Scintillation | 343 |
| (agonist | recombinant | | | | | | (1 μM) | | counting | |
| radioligand) | (CHO cells) | | | | | | | | | |
| V2 (h) | human | [3H]AVP | 0.3 | nM | 0.76 | nM | AVP | 120 min RT | Scintillation | 343 |
| (agonist | recombinant | | | | | | (1 μM) | | counting | |
| radioligand) | (CHO cells) | | | | | | | | | |

Ion channels

| | | | | | | | | | | |
|---|---|---|---|---|---|---|---|---|---|---|
| Sodium | rat brain | Batrachotoxin | 15 | nM | 8.9 | nM | Veratridine | 60 minutes | MS | 28 |
| Channel Site2 | | | | | | | (1 mM) | at 37° C. | | |
| (Non- | | | | | | | | | | |
| selective) Rat | | | | | | | | | | |
| Ion Channel | | | | | | | | | | |
| Batrachotoxin | | | | | | | | | | |
| Mass | | | | | | | | | | |
| Spectrometry | | | | | | | | | | |
| Binding | | | | | | | | | | |
| Glutamate | rat brain | MDL 105.519 | 2 | nM | 1.7 | nM | 5,7 | 60 min à 0° C. | MS | 219 |
| (Non- | | | | | | | dichlorokynurenic | | | |
| Selective) Rat | | | | | | | acid (10 μM) | | | |
| Ion Channel | | | | | | | | | | |
| Glycine | | | | | | | | | | |
| (Strychnine- | | | | | | | | | | |
| Insensitive) | | | | | | | | | | |
| Mass | | | | | | | | | | |
| Spectrometry | | | | | | | | | | |
| Binding | | | | | | | | | | |
| Cav1.2 (L- | rat brain | Diltiazem | 20 | nM | 9.6 | nM | D600 | 60 min RT | MS | 212 |
| type) Rat | | | | | | | | | | |
| Calcium Ion | | | | | | | | | | |
| Channel | | | | | | | | | | |
| (Diltiazem | | | | | | | | | | |
| Site) Mass | | | | | | | | | | |
| Spectrometry | | | | | | | | | | |
| Binding | | | | | | | | | | |
| BZD (central) | rat cerebral | [3H]flunitrazepam | 0.4 | nM | 2.1 | nM | diazepam | 60 min 4° C. | Scintillation | 227 |
| (agonist | cortex | | | | | | (3 μM) | | counting | |
| radioligand) | | | | | | | | | | |
| AMPA | rat cerebral | [3H]AMPA | 8 | nM | 82 | nM | L-glutamate | 60 min 4° C. | Scintillation | 166 |
| (agonist | cortex | | | | | | (1 mM) | | counting | |
| radioligand) | | | | | | | | | | |
| kainate | rat cerebral | [3H]kainic acid | 5 | nM | 19 | nM | L-glutamate | 60 min 4°° C. | Scintillation | 160 |
| (agonist | cortex | | | | | | (1 mM) | | counting | |
| radioligand) | | | | | | | | | | |
| NMDA | rat cerebral | [3H]CGP | 5 | nM | 23 | nM | L-glutamate | 60 min 4° C. | Scintillation | 221 |
| (antagonist | cortex | 39653 | | | | | (100 μM) | | counting | |
| radioligand) | | | | | | | | | | |
| PCP | rat cerebral | [3H]TCP | 10 | nM | 13 | nM | MK 801 | 120 min 37° C. | Scintillation | 257 |
| (antagonist | cortex | | | | | | (10 μM) | | counting | |
| radioligand) | | | | | | | | | | |
| 5-HT3 (h) | human | [3H]BRL 43694 | 0.5 | nM | 1.15 | nM | MDL 72222 | 120 min RT | Scintillation | 109 |
| (antagonist | recombinant | | | | | | (10 μM) | | counting | |
| radioligand) | (CHO cells) | | | | | | | | | |
| Ca2+ channel | rat cerebral | [3H]nitrendipine | 0.25 | nM | 0.27 | nM | nitrendipine | 90 min RT | Scintillation | 996 |
| (L | cortex | | | | | | (1 μM) | | counting | |
| dihydropyridine | | | | | | | | | | |
| site) | | | | | | | | | | |
| (antagonist | | | | | | | | | | |
| radioligand) | | | | | | | | | | |
| Ca2+ channel | rat cerebral | [3H]D888 | 3 | nM | 3 | nM | D 600 | 120 min RT | Scintillation | 194 |
| (L verapamil | cortex | | | | | | (10 μM) | | counting | |
| site) | | | | | | | | | | |
| (phenylalkylamine) | | | | | | | | | | |
| (antagonist | | | | | | | | | | |
| radioligand) | | | | | | | | | | |
| Ca2+ channel | rat cerebral | [125I]ω- | 0.001 | nM | 0.0007 | nM | ω-conotoxin | 30 min RT | Scintillation | 259 |
| (N) | cortex | conotoxin | | | | | GVIA | | counting | |
| (antagonist | | GVIA | | | | | (10 nM) | | | |
| radioligand) | | | | | | | | | | |
| SKCa channel | rat cerebral | [125I]apamin | 0.007 | nM | 0.007 | nM | apamin | 60 min 4° C. | Scintillation | 112 |
| (antagonist | cortex | | | | | | (100 nM) | | counting | |
| radioligand) | | | | | | | | | | |

Transporters

| | | | | | | | | | | |
|---|---|---|---|---|---|---|---|---|---|---|
| norepinephrine | human | [3H]nisoxetine | 1 | nM | 2.9 | nM | desipramine | 120 min 4° C. | Scintillation | 180 |
| transporter (h) | recombinant | | | | | | (1 μM) | | counting | |
| (antagonist | (CHO cells) | | | | | | | | | |
| radioligand) | | | | | | | | | | |
| dopamine | human | [3H]BTCP | 4 | nM | 4.5 | nM | BTCP | 120 min 4° C. | Scintillation | 190 |
| transporter (h) | recombinant | | | | | | (10 μM) | | counting | |
| (antagonist | (CHO cells) | | | | | | | | | |
| radioligand) | | | | | | | | | | |
| GABA | rat cerebral | [3H]GABA | 10 | nM | 4600 | nM | GABA | 30 min RT | Scintillation | 214 |
| transporter | | (+10 μM | | | | | (1 mM) | | counting | |
| (antagonist | | isoguvacine) | | | | | | | | |
| radioligand) | cortex | (+10 μM | | | | | | | | |
| | | baclofen) | | | | | | | | |
| choline | human | [3H]hemicholinium- | 3 | nM | 3.9 | nM | hemicholinium-3 | 60 min RT | Scintillation | 648 |
| transporter | recombinant | 3 | | | | | (10 μM) | | counting | |
| (CHT1) (h) | (CHO cells) | | | | | | | | | |
| (antagonist | | | | | | | | | | |
| radioligand) | | | | | | | | | | |
| 5-HT | human | [3H]imipramine | 2 | nM | 1.7 | nM | imipramine | 60 min RT | Scintillation | 566 |
| transporter | recombinant | | | | | | (10 μM) | | counting | |
| (h) | (CHO cells) | | | | | | | | | |
| (antagonist | | | | | | | | | | |
| radioligand) | | | | | | | | | | |

Other enzymes

| | | | | | | | | | | |
|---|---|---|---|---|---|---|---|---|---|---|
| MAO-A | rat cerebral | [3H]Ro | 10 | nM | 14 | nM | clorgyline | 60 min 37° C. | Scintillation | 36 |
| (antagonist | cortex | 41-1049 | | | | | (1 μM) | | counting | |
| radioligand) | | | | | | | | | | |

### 9.1.2. In Vitro Pharmacology: Enzyme and Uptake Assays

| | | | | | | |
|---|---|---|---|---|---|---|
| | | Substrate/ | | Measured | Detection | |
| Assay | Source | Stimulus/Tracer | Incubation | Component | Method | Bibl. |

Customized assays

| | | | | | | |
|---|---|---|---|---|---|---|
| Abl (ABL1) Human | recombinant humain | 33P | 40 min RT | ATP + | Scintillation | 1646, 1645 |
| TK Kinase | (cellules d'insectes) | | | EAIYAAPFAKKK | counting | |
| Enzymatic | | | | (50 μM) | | |
| Radiometric [Km | | | | | | |
| ATP | | | | | | |
| CaMK2alpha | recombinant humain | 33P | 40 min RT | ATP + | Scintillation | 1646, 1645 |
| (CAMK2A) (Active) | (cellules d'insectes) | | | KKLNRTLSFAEPG | counting | |
| Human CAMK | | | | (250 μM) | | |
| Kinase Enzymatic | | | | | | |
| Radiometric [Km | | | | | | |
| ATP | | | | | | |
| CDK2/CyclinA | recombinant humain | 33P | 40 min RT | ATP + histone H1 | Scintillation | 1646, 1645 |
| Human CMGC | (cellules d'insectes) | | | (0.1 mg/ml) | counting | |
| Kinase Enzymatic | | | | | | |
| Radiometric [Km | | | | | | |
| ATP | | | | | | |
| ERK2 (MAPK1) | E. coli | 0.33 mg/mL | 40 min RT | ATP + myelin basic | Scintillation | 1645, 1646 |
| Human CMGC | | MBP/33P | | protein (0.33 mg/ml) | counting | |
| Kinase Enzymatic | | | | | | |
| Radiometric [Km | | | | | | |
| ATP | | | | | | |
| Flt-1 (VEGFR1) | recombinant humain | 33P | 40 min RT | ATP + | Scintillation | 1645, 1646 |
| (FLT1) Human RTK | (cellules d'insectes) | | | KKKSPGEYVNIEFG | counting | |
| Kinase Enzymatic | | | | (250 μM) | | |
| Radiometric [Km | | | | | | |
| ATP] | | | | | | |
| Fyn Human TK | recombinant humain | 33P | 40 min RT | ATP + | Scintillation | 1645, 1646 |
| Kinase Enzymatic | (cellules d'insectes) | | | KVEKIGEGTYGVVYK | counting | |
| Radiometric [Km | | | | (250 μM) | | |
| ATP | | | | | | |
| LYN Human TK | recombinant humain | 0.1 mg/mL poly | 40 min RT | ATP + poly(Glu, Tyr) | Scintillation | 1645 |
| Kinase Enzymatic | (cellules d'insectes) | (Glu, Tyr)/ | | 4:1 (0.1 mg/ml) | counting | |
| Radiometric [Km | | 33P | | | | |
| ATP] | | | | | | |
| p38alpha | E. coli | 0.33 mg/mL MBP/ | 40 min RT | ATP + myelin basic | Scintillation | 1646, 1645 |
| (SAPK2A) Human | | 33P | | protein (0.33 mg/ml) | counting | |
| CMGC Kinase | | | | | | |
| Enzymatic | | | | | | |
| Radiometric [Km | | | | | | |
| ATP] | | | | | | |
| ZAP70 Human TK | recombinant humain | 33P | 40 min RT | ATP + poly(Glu, Tyr) | Scintillation | 1645, 1646 |
| Kinase Enzymatic | (cellules d'insectes) | | | 4:1 (0.1 mg/mL) | counting | |
| Radiometric Assay | | | | | | |
| [Km ATP] | | | | | | |
| IR (INSR) Human | recombinant humain | 33P | 40 min RT | ATP + | Scintillation | 1646, 1645 |
| RTK Kinase | (cellules d'insectes) | | | KKSRGDYMTMQIG | counting | |
| Enzymatic | | | | (250 μM) | | |
| Radiometric [Km | | | | | | |
| ATP] | | | | | | |

Other enzymes

| | | | | | | |
|---|---|---|---|---|---|---|
| COX1(h) | human recombinant | Arachidonic acid | 3 min RT | Resorufin (oxydized | Fluorimetry | 1480 |
| | | (3 μM) + ADHP | | ADHP) | | |
| | | (25 μM) | | | | |
| COX2(h) | human recombinant | arachidonic acid | 5 min RT | Resorufin (oxydized | Fluorimetry | 1480 |
| | (Sf9 cells) | (1.2 μM)+ ADHP | | ADHP) | | |
| | | (25 μM) | | | | |
| inducible NOS | mouse recombinant | L-arginine | 120 min 37° C. | NO— | Photometry | 236 |
| | (E. coli) | (100 μM) | | | | |
| PDE2A1 (h) | human recombinant | [3H]cAMP + cAMP | 10 min RT | [3H]5′AMP | Scintillation | 1399 |
| | (Sf21 cells) | (10 μM) | | | counting | |
| PDE3B (h) | human recombinant | [3H]cAMP + cAMP | 20 min RT | [3H]5′AMP | Scintillation | 1399 |
| | (Sf9 cells) | (0.5 μM) | | | counting | |
| PDE4D2 (h) | human recombinant | [3H]cAMP + cAMP | 20 min RT | [3H]5′AMP | Scintillation | 1399 |
| | (Sf9 cells) | (0.5 μM) | | | counting | |
| PDE5 (h) | human platelets | [³H]cGMP + | 30 min RT | [3H]5′GMP | Scintillation | 263 |
| (non-selective) | | cGMP (1 μM) | | | counting | |
| PDE6 | bovine retina | [³H]cGMP + | 60 min RT | [3H]5′GMP | Scintillation | 306 |
| (non-selective) | | cGMP (2 μM) | | | counting | |
| ACE-2 (h) | human recombinant | MCa-Tyr-Val-Ala- | 20 min RT | Mca peptides | Fluorimetry | 802 |
| | (murine cells) | Asp-Pro-Ala-Lys- | | | | |
| | | (DNP)-OH | | | | |
| | | (10 μM) | | | | |
| ACE (h) | human recombinant | Abz-FRK(Dnp)-P- | 30 min 37° C. | Abz-Phe-Arg | Fluorimetry | 1128 |
| | | OH | | | | |
| | | (15 μM) | | | | |
| BACE-1 (h) | human recombinant | Mca-S-E-V-N-L-D-A- | 60 min RT | Mca-S-E-V-N-L-NH2 | Fluorimetry | 462 |
| (beta-secretase) | (mammalian cells) | E-F-R-K(Dnp)-R-R- | | | | |
| | | NH2 (6 μM) | | | | |
| caspase-3 (h) | human recombinant | benzyloxycarbonyl- | 30 min RT | AFC | Fluorimetry | 476 |
| | (E. coli) | Asp-Glu-Val-Asp- | | | | |
| | | AFC | | | | |
| | | (3.6 μM) | | | | |
| HIV-1 protease | protein viral | antranilyl-HIV | 20 min 37° C. | N-terminal tripeptide | Fluorimetry | 244 |
| | recombinant (E. coli) | (75 μM) | | | | |
| MMP-1 (h) | human recombinant | DNP-Pro-Cha-Gly- | 20 min 37° C. | Cys(Me)-His-Ala- | Fluorimetry | 342 |
| | (E. coli) | Cys(Me)-His-Ala-Lys(n- | | Lys(n-Me-Abz)-NH2 | | |
| | | Me-Abz)-NH2 (10 μM) | | | | |
| guanylyl cyclase | human recombinant | GTP | 10 min RT | cGMP | HTRF | 1076 |
| (h) (activator | | (10 μM) | | | | |
| effect) | | (100 μM SNP | | | | |
| | | for control) | | | | |
| acetylcholinesterase | human recombinant | Acetylthiocholine | 30 min RT | 5 thio 2 nitrobenzoic | Photometry | 63 |
| (h) | (HEK-293 cells) | (400 μM) | | acid | | |
| xanthine oxidase/ | purified xanthine | hypoxanthine (10 μM) | 10 min RT | O2— + uric acid | Photometry | 153 |
| superoxide O2— | oxidase | | | | | |
| scavenging | from bovine milk | | | | | |
| ATPase (Na+/K+) | porcine cerebral | ATP | 60 min 37° C. | Pi | Photometry | 71 |
| | cortex | (2 mM) | | | | |

### 10. BIBLIOGRAPHY

3. Aharony, D. et al. (1993), Mol. Pharmacol., 44:356-363.
13. Berkhout, T. A. et al. (1997), J. Biol. Chem., 272:16404-16413.
26. Brockhaus, M. et al. (1990), Proc. Natl. Acad. Sci. U.S.A., 87:3127-3131.
28. Brown, G. B. (1986), J. Neurosci., 6:2064-2070.
30. Buchan, K. W. et al. (1994), Brit. J. Pharmacol., 112:1251-1257.
36. Cesura, A. M. et al. (1990), Mol. Pharmacol., 37:358-366.
50. Couvineau, A. et al. (1985), Biochem. J., 231:139-143.
56. Devedjian, J. C. et al. (1994), Eur. J. Pharmacol., 252:43-49.
59. Dorje, F. et al. (1991), J. Pharmacol. Exp. Ther., 256:727-733.
63. Ellman, G. L. et al. (1961), Biochem. Pharmacol., 7:88-95.
71. Fiske, C. M. and Subbarow, Y. (1925), J. Biol. Chem., 66:375-400.
87. Grandy, D. K. et al. (1989), Proc. Natl. Acad. Sci. U.S.A., 86:9762-9766.
104. Heuillet, E. et al. (1993), J. Neurochem., 60:868-876.
109. Hope, A. G. et al. (1996), Brit. J. Pharmacol., 118:1237-1245.
112. Hugues, M. et al. (1982), J. Biol. Chem., 257:2762-2769.
134. Lee, Y. M. et al. (1993), J. Biol. Chem., 268:8164-8169.
141. Luthin, D. R. et al. (1995), Mol. Pharmacol., 47:307-313.
145. Mackenzie, R. G. et al. (1994), Eur. J. Pharmacol., 266:79-85.
153. McCord, J. K. and Fridovich, I. (1969), J. Biol. Chem., 244:6049-6055.
160. Monaghan, D. T. and Cotman, C. W. (1982), Brain Res., 252:91-100.
161. Monsma, F. J. et al. (1993), Mol. Pharmacol., 43:320-327.
164. Mulheron, J. G. et al. (1994), J. Biol. Chem., 269:12954-12962.
165. Munro, S. et al. (1993), Nature, 365:61-65.
166. Murphy, D. E. et al. (1987), Neurochem. Res., 12:775-781.
180. Pacholczyk, T. et al. (1991), Nature, 350:350-354.
186. Pickering, D. S. and Niles, L. P. (1990), Eur. J. Pharmacol., 175:71-77.
190. Pristupa, Z. B. et al. (1994), Mol. Pharmacol., 45:125-135.
194. Reynolds, I. J. et al. (1986), J. Pharmacol. Exp. Ther., 237:731-738.
198. Rivkees, S. A. et al. (1995), J. Biol. Chem., 270:20485-20490.
206. Salvatore, C. A. et al. (1993), Proc. Natl. Acad. Sci. U.S.A., 90:10365-10369.
211. Schioth, H. B. et al. (1997), Neuropeptides, 31:565-571.
212. Schoemaker, H. and Langer, S. Z. (1985), Eur. J. Pharmacol., 111:273-277.
214. Shank, R. P. et al. (1990), J. Neurochem., 54:2007-2015.
217. Shen, Y. et al. (1993), J. Biol. Chem., 268:18200-18204.
219. Baron, B. M. et al. (1996), J. Pharmacol. Exp. Ther., 279:62-68.
221. Sills, M. A. et al. (1991), Eur. J. Pharmacol., 192:19-24.
222. Simonin, F. et al. (1995), Proc. Natl. Acad. Sci. U.S.A., 92:7006-7010.
227. Speth, R. C. et al. (1979), Life Sci., 24:351-358.
229. Stehle, J. H. et al. (1992), Mol. Endocrinol., 6:384-393.
236. Tayeh, M. A. and Marletta, M. A. (1989), J. Biol. Chem., 264:19654-19658.
244. Toth, M. V. and Marshall, G. R. (1990), Int. J. Protein Res., 36:544-550.
248. Tsuzuki, S. et al. (1994), Biochem. Biophys. Res. Commun., 200:1449-1454.
257. Vignon, J. et al. (1986), Brain Res., 378:133-141.
259. Wagner, J. A. et al. (1988), J. Neurosci., 8:3354-3359.
260. Wang, J. B. et al. (1994), FEBS Lett., 338:217-222.
263. Weishaar, R. E. et al. (1986), Biochem. Pharmacol., 35:787-800.
281. Zhou, Q. Y. et al. (1990), Nature, 347:76-80.
283. Clark, A. F. et al. (1996), Invest. Ophtalmol. Vis. Sci., 37:805-813.
285. Feighner, S. D. et al. (1999), Science, 284:2184-2188.
287. Mantey, S. A. et al. (1997), J. Biol. Chem., 272:26062-26071.
288. Bryant, H. U. et al. (1996), Life Sci., 15:1259-1268.
296. Rohrer, L. et al. (1993), Proc. Natl. Acad. Sci. U.S.A., 90:4196-4200.
306. Ballard, A. S. et al. (1998), J. Urol., 159:2164-2171.
309. Mialet, J. et al. (2000), Brit. J. Pharmacol., 129:771-781.
342. Bickett, D. A. et al. (1993), Anal. Biochem., 212:58-64.
343. Tahara, A. et al. (1998), Brit. J. Pharmacol., 125:1463-1470.
346. Pruneau, D. et al. (1998), Brit. J. Pharmacol., 125:365-372.
390. Siegrist, W. et al. (1988), J. Recep. Res., 8:323-343.
391. Wieland, H. A. et al. (1995), J. Pharmacol. Exp. Ther., 275:143-149.
462. Ermolieff, J. et al. (2000), Biochemistry, 39:12450-12456.
476. Mittl, P. R. E. et al. (1997), J. Biol. Chem., 272:6539-6547.
492. Smit, M. J. et al. (1996), Brit. J. Pharmacol., 117:1071-1080.
498. Zava, D. T. et al. (1979), Endocrinology, 104:1007-1012.
501. Simonin, F. et al. (1994), Mol. Pharmacol., 46:1015-1021.
508. Green, A. et al. (2000), Brit. J. Pharmacol., 131:1766-1774.
524. Lukas, R. J. (1986), J. Neurochem., 46:1936-1941.526.
526. Mac Donald, D. et al. (2000), Mol. Pharmacol., 58:217-225.
531. Fukunaga, K. et al. (2001), J. Biol. Chem., 276:43025-43030.
540. Leurs, R. et al. (1994), Brit. J. Pharmacol., 112:847-854.
541. Fuchs, S. et al. (2001), Mol. Med., 7:115-124.
542. Langin, D. et al. (1989), Eur. J. Pharmacol., 167:95-104.
546. Peralta, E. G. et al. (1987), Embo. J., 6:3923-3929.
548. Levin, M. C. et al. (2002), J. Biol. Chem., 277:30429-30435.
557. Palchaudhuri, M. R. et al. (1998), Eur. J. Biochem., 258:78-84.
562. Bignon, E. et al. (1999), J. Pharmacol. Exp. Ther. 289:742-751.
563. Lovenberg, T. W. et al. (1999), Mol. Pharmacol., 55:1101-1107.
566. Tatsumi, M. et al. (1999), Eur. J. Pharmacol., 368:277-283.
567. Ferry, G. et al. (2001), Eur. J. Pharmacol., 417:77-89.
571. Choi, D. S. et al. (1994), FEBS Lett., 352:393-399.
618. Martin, V. et al. (2001), Biochem. Pharmacol., 62:1193-1200.
622. Maguire, J. J. et al. (2000), Brit. J. Pharmacol., 131:441-446.
624. Chicchi, G. G. et al. (1997), J. Biol. Chem., 272:7765-7769.
631. Liu, C. et al. (2001), J. Pharmacol. Exp. Ther., 299:121-130.
639. Witt-Enderby, P. A. and Dubocovich, M. L. (1996), Mol. Pharmacol., 50:166-174.
648. Apparsundaram, S. et al. (2000), Biochem. Biophys. Res. Commun., 276:862-867.
657. Rinaldi-Carmona, M. et al. (1996), J. Pharmacol. Exp. Ther., 278:871-878.
701. Ford, A. P. D. W. et al. (1997), Brit. J. Pharmacol., 121:1127-1135.
761. Patel, C. Y. and Srikant, C. B. (1994), Endocrinology, 135:2814-2817.
776. Le, M. T. et al. (2005), Eur. J. Pharmacol., 513:35-45.
777. Wurch, T. et al. (1997), J. Neurochem., 68:410-418.
781. Abramovitz, M. et al. (2000), Biochem. Biophys. Acta., 1483:285-293.
794. Joseph, S. S. et al. (2004), Naun.-Sch. Arch. Pharm., 369:525-532.
802. Huang, L. et al. (2003), J. Biol. Chem., 278:15532-15540.
846. Katugampola, S. D. et al. (2001), Brit. J. Pharmacol., 132:1255-1260.
855. Boehm, F. M. et al. (1994), J. Med. Chem., 37:408-414.
897. Schwinn, D. A. et al. (1990), J. Biol. Chem., 265:8183-8189.
996. Gould, R. J. et al. (1982), Proc. Natl. Acad. Sci. USA., 79:3656-3660.
1076. Lee, Y. C. et al. (2000), Proc. Natl. Acad. Sci. USA, 20:10763-10768.
1084. Gopalakrishnan, M. et al. (1996), J. Pharmacol. Exp. Ther., 276:289-297.
1096. Wang, X. K. (2001), Acta. Pharmacol. Sin., 22:521-523.
1128. Fernandes, T. et al. (2010), Braz J Med Biol Res., 43:837-842
1136. GANAPATHY M E. et al. (1999), JPET, 289:251-260
1222. Mutel, V. et al. (2000), J. Neurochem., 75:2590-2601
1277. Feve B, Elhadri K, Quignard-Boulange A and Pairault J (1994), Feve B et al. Proc Natl Acad Sci USA. 91:5677, 1994.
1280. Obourn J D, Koszewski N J and Notides A C (1993), Obourn J D et al. Biochemistry 32(24):6229, 1993.
1399. Maurice D. H. et al. (2014), Nat Rev Drug Discov., 13:290-314
1451. Maier D L, Sobotka-Briner C, Ding M, Powell M E, Jiang Q, Hill G, Heys J R, Elmore C S, Pierson M E and Mrzljak L (2009), Maier D L et al. J Pharmacol Exp Ther. 330(1):342, 2009.
1480. Pattaraporn Vanachayangkul and William H. Tolleson (2012), Hindawi Publishing Corporation, Enzyme Research, Volume 2012, Article ID 416062, 7
1578. J W A van der Hoorn, D Linden, U Lindahl, M E A Bekkers, M Voskuilen, R Nilsson, J Oscarsson, E L Lindstedt and H M G Princen (2010), British Journal of Pharmacology, 162:1553-1563
1588.1. Mogil J. S. and Pasternak G. W. (2001), Pharmacol. Rev. 53:381-415.
1645. Davies S P, Reddy H, Caivano M and Cohen P, Biochem J. 351(Pt 1): 95, 2000.
1646. Gao Y, Davies S P, Augustin M, Woodward A, Patel U A, Kovelman R, Harvey K J, Biochem J. 451, 313-28, 2013.
1664. Holland, K. D. et al. (1993), Brain Research, 615:170-174

### Analysis and Expression of Results

### In Vitro Pharmacology: Binding Assays

The results are expressed as a percent of control specific activity

$$Measured specific binding / Control specific binding * 100 and as a percent inhibition of control specific binding$$

$$100 - ( Measured specific binding / Control specific binding * 100 ) obtained in the presence of WB 3.$$

The IC₅₀ values (concentration causing a half-maximal inhibition of control specific activity), EC₅₀ values (concentration producing a half-maximal increase in control basal activity), and Hill coefficients (nH) were determined by non-linear regression analysis of the inhibition/concentration-response curves generated with mean replicate values using Hill equation curve fitting

$$Y = D + [ A - D / 1 + {( C / {C}_{50} )}^{nH} ]$$

where Y=specific activity, A=left asymptote of the curve, D=right asymptote of the curve, C=compound concentration, C50=IC50 or EC50, and nH=slope factor.

This analysis was performed using software developed at Cerep (Hill software) and validated by comparison with data generated by the commercial software SigmaPlot® 4.0 for Windows® (© 1997 by SPSS Inc.).

The inhibition constants (Ki) were calculated using the Cheng Prusoff equation

$$Ki = {IC}_{50} / ( 1 + L / {K}_{D} )$$

where L=concentration of ligand in the assay, and KD=affinity of the ligand for the receptor.

### In Vitro Pharmacology: Enzyme and Uptake Assays

The results are expressed as a percent of control specific activity

$$Measured specific activity / Control specific activity * 100 and as a percent inhibition of control specific activity$$

$$100 - ( Measured specific activity / Control specific activity * 100 ) obtained in the presence of WB 3.$$

The IC₅₀ values (concentration causing a half-maximal inhibition of control specific activity), EC₅₀ values (concentration producing a half-maximal increase in control basal activity), and Hill coefficients (nH) were determined by non-linear regression analysis of the inhibition/concentration-response curves generated with mean replicate values using Hill equation curve fitting

$$Y = D + [ A - D / 1 + {( C / {C}_{50} )}^{nH} ]$$

where Y=specific activity, A=left asymptote of the curve, D=right asymptote of the curve, C=compound concentration, C50=IC50 or EC50, and nH=slope factor.

This analysis was performed using software developed at Cerep (Hill software) and validated by comparison with data generated by the commercial software SigmaPlot® 4.0 for Windows® (© 1997 by SPSS Inc.).

Comparison of WB3 with Other MMCs

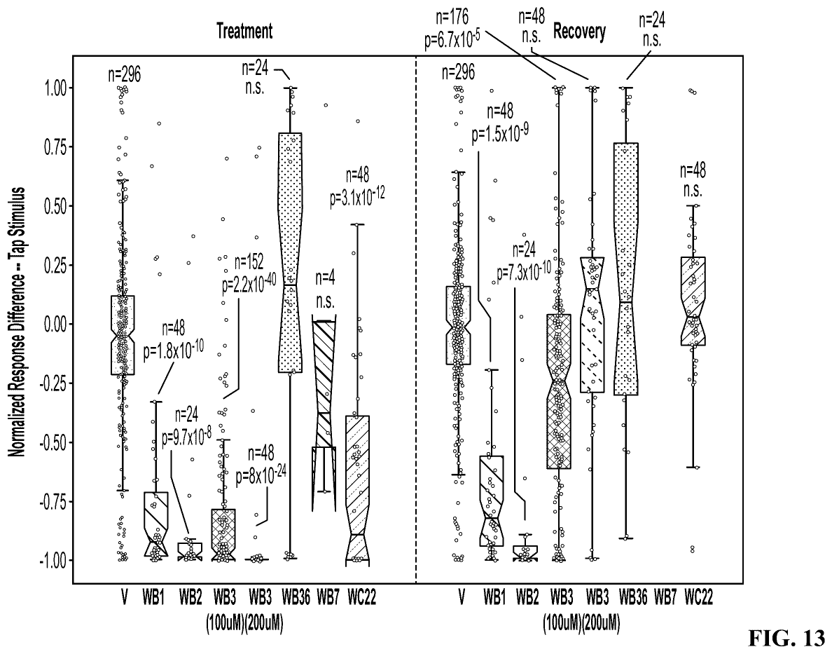

WB3 was compared against other Mobility Modulating Compounds (MMCs) in mobility assays, arousal (“tap”), and pain assays (via Optovin or electric shock). Results are shown in FIGS. 11, 13, and 14 and discussed below.

MMCs included WB1 (SNC-80), WB2, WB7, WB36, and WB22 (Donepezil):

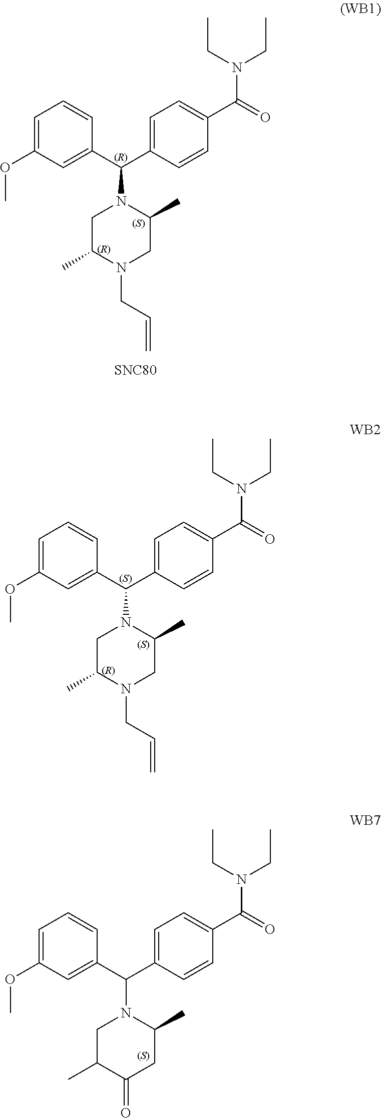

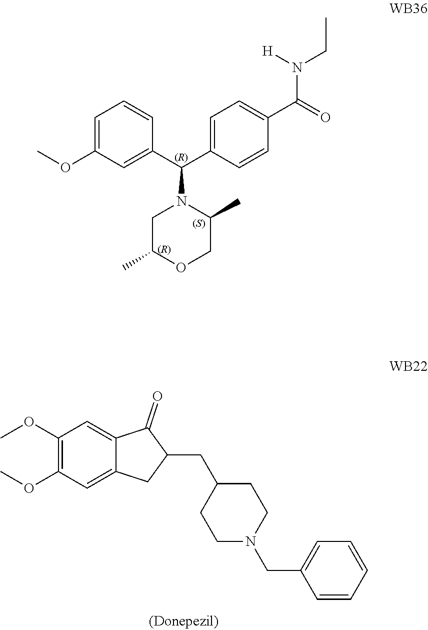

As shown in FIG. 11, WB3 showed dose-dependent fast induction and slower recovery in comparison to WB7 and WB36. The WB1 compound was least effective in suppressing movement and shows slow recovery, and WB2 showed medium induction speed and slow recovery, WC22 showed relatively fast induction but was the slowest to recover.

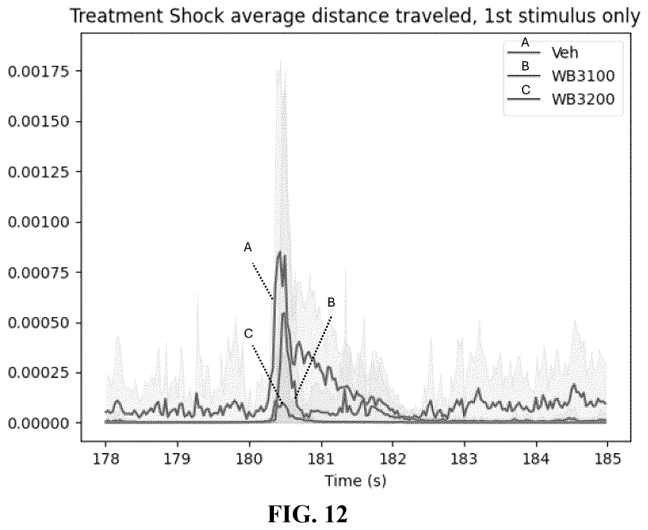

As shown in FIG. 12, all MMCs, except WB36 and WB7, significantly reduced response to aversive tap, despite their effectiveness in suppressing mobility. WB3 showed the best combination of response suppression and recovery, while WB1 and WB2 showed strong suppression but showed a prolonged effect after wash.

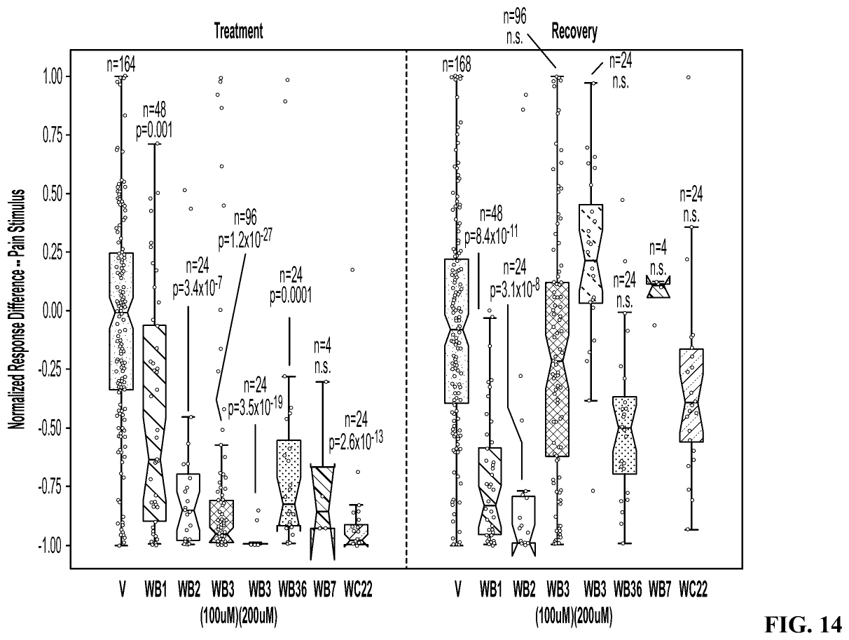

As shown in FIG. 14, all MMCs significantly reduce response to pain stimulus evoked by UV-activated Optovin (TRPA1 channel agonist). WB3 showed the best combination of response suppression and recovery, while WB1, WB2 and WB36 each exhibited a prolonged effect after wash.

Comparison of WB3 with SOA Anesthetics

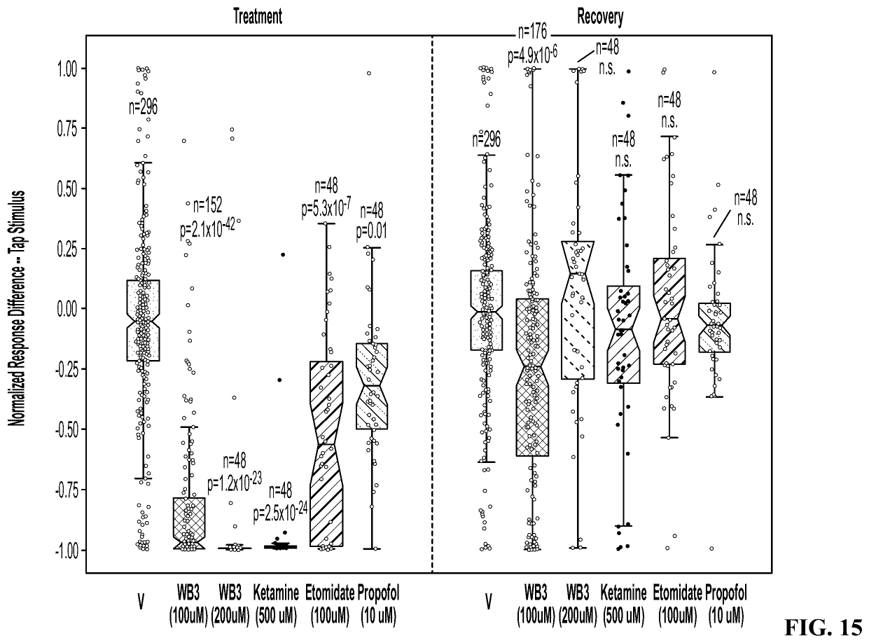

WB3 was compared against state-of-the-art (SOAs) aesthetics in mobility assays, arousal (“tap”), and pain assays (via Optovin or electric shock). Results are shown in FIGS. 15-17 and discussed below. SOAs included ketamine, etomidate, and propofol.

As shown in FIG. 15, WB3 suppressed response to tap, and its effect was fully reversed 3 hours after wash. WB3 showed a dose-dependent suppression in responses to tap and at 200 μM, the effect size was comparable to ketamine at 500 μM. Etomidate and propofol did not suppress the tap response as much as WB3 and ketamine did.

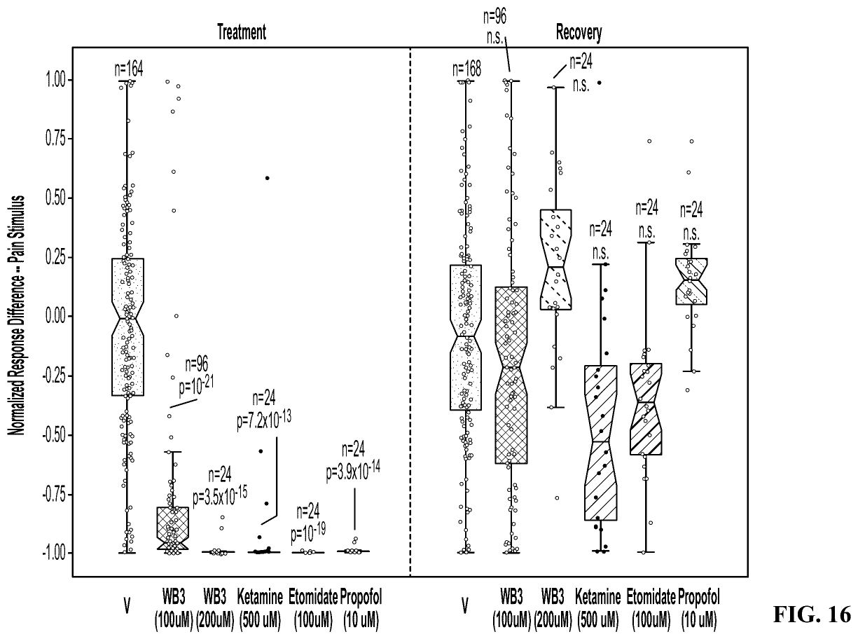

As shown in FIG. 16, WB3 suppressed response to Optovin, and its effect was fully reversed 3 hours after wash. WB3 treated tadpoles showed a significantly better recovery profile compared to those treated with ketamine and etomidate.

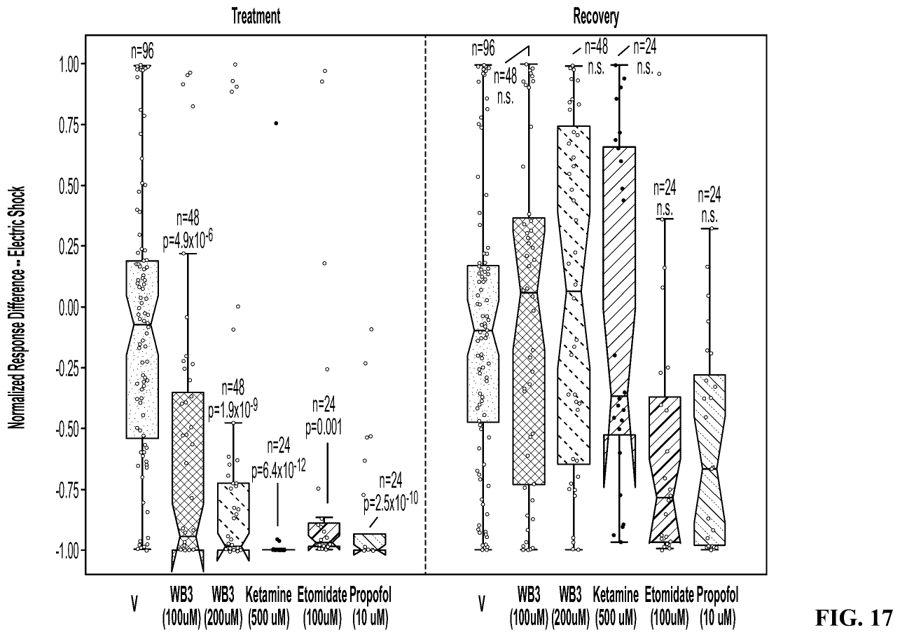

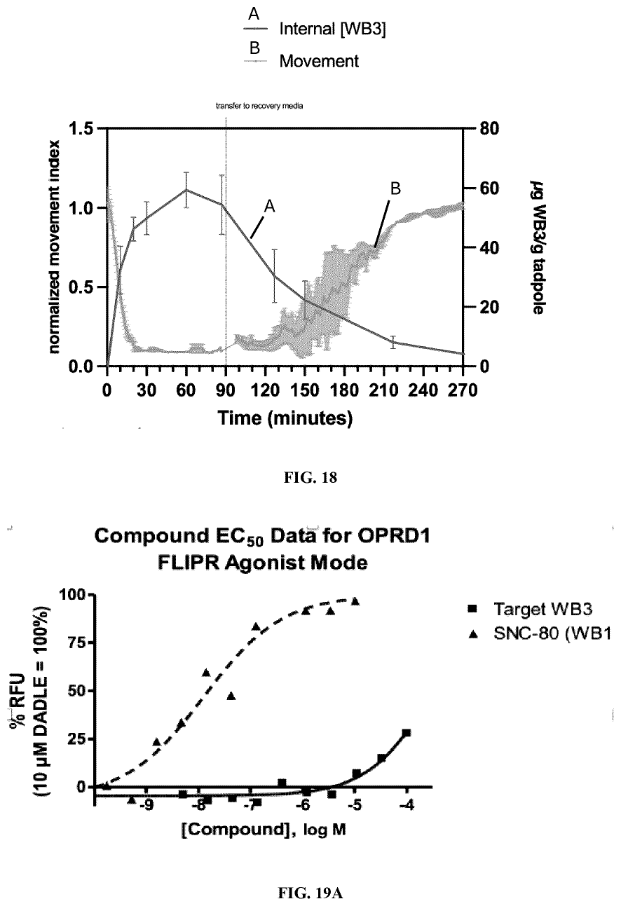

As shown in FIG. 17, WB3 suppressed response to shock, and its effect was fully reversed 3 hours after wash. WB3 showed a dose-dependent suppression in responses to electric shocks. WB3 treated tadpoles show significantly better recovery profile compared to those treated with etomidate and propofol.

Although WB3 had a slower induction speed compared to ketamine and other SOAs, it performs similarly or better than ketamine at all other measures, especially at the higher dose. Additionally, WB3 (high dose) showed improved recovery of Optovin-pain response.

| | WB3 | WB3 | Etomidate | Propofol |
|---|---|---|---|---|
| Features | (100 μM) | (200 μM) | (100 μM) | (10 μM) |
| Movement | = | = | = | = |
| Suppression | | | | |
| Induction Speed | Slower | Slower | = | = |
| Recovery Speed | = | Slower | Faster | Slower |
| Movement after | = | = | Overactivity | = |
| Recovery | | | | |
| Arousal | = | = | Less | Less |
| Suppression | | | Suppressive | Suppressive |
| Arousal | = | = | = | = |
| Recovery | | | | |
| Pain | = | = | = | = |
| Suppression | | | | |
| (Optovin) | | | | |
| Pain Response | = | Faster | = | Faster |
| Recovery | | | | |
| (Optovin) | | | | |
| Pain | Less | = | Less | = |
| Suppression | Suppressive | | Suppressive | |
| (Optovin) | | | | |
| Pain Response | = | == | = | = |
| Recovery | | | | |
| (Shock) | | | | |

The symbol “=” indicates effect is not significantly different from ketamine at 500 μM at p = 0.05 level.

### Cell Based Assays:

Fluorescence Imaging Plate Reader (FLIPR) assay. Reading intracellular calcium activity in CHO/ORPD/Galpha15 cell line.

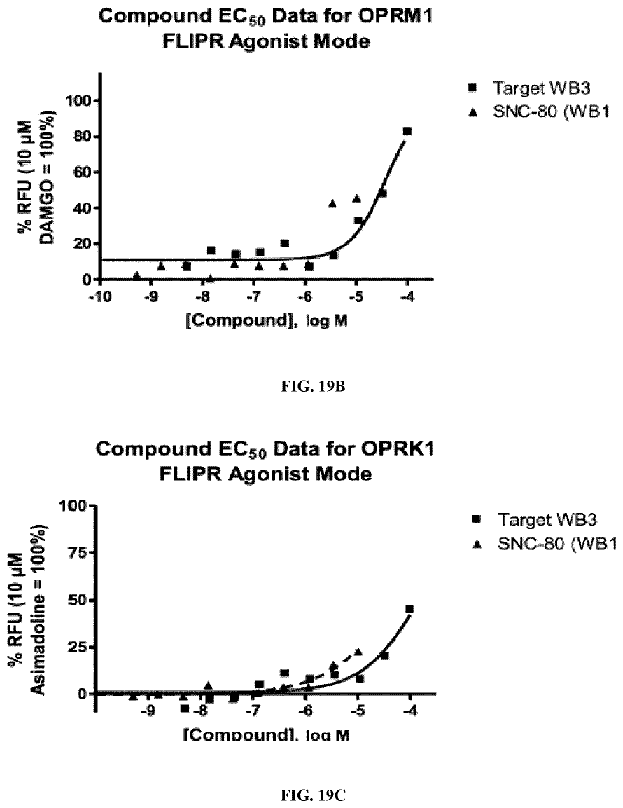

Cell-based assays showed that delta opioid receptor activity was shut down in WB3. Results are shown in FIGS. 19A-19C. The assays demonstrated that unlike SNC-80, WB3 does not activate delta opioid receptors and like SNC-80, it does not bind to Kappa and Mu opioid receptors.

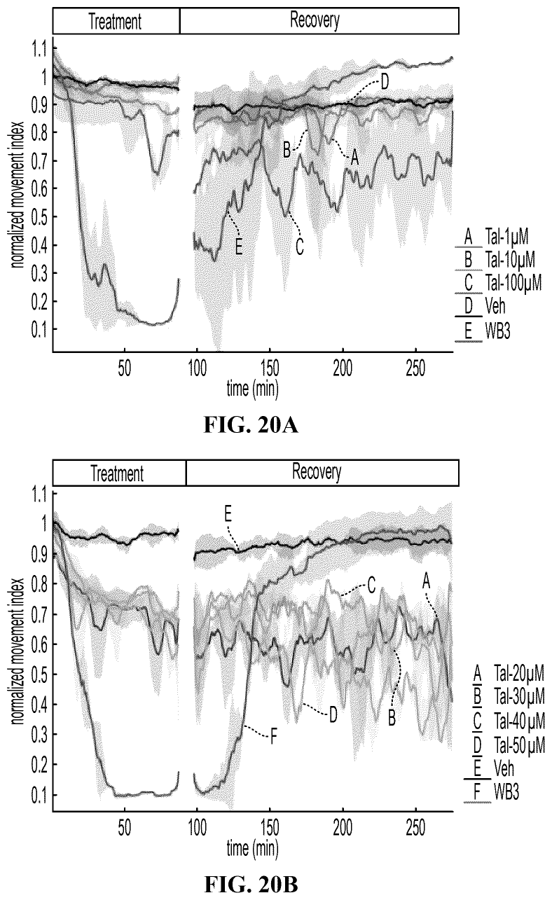

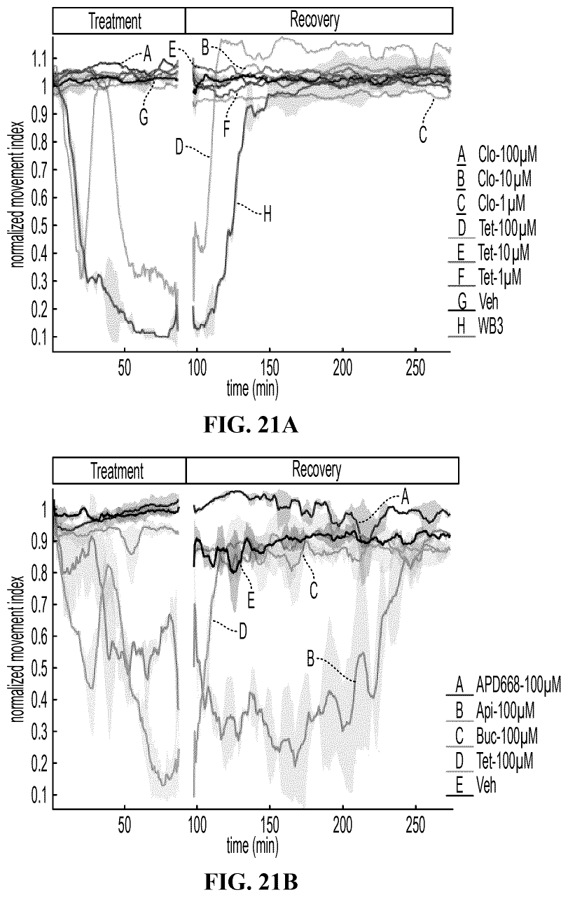

WB3 was tested in two safety and off-target screens against ˜900 targets (receptors, kinases, proteases, etc.). Results indicated that WB3 does not directly bind to GABA-A receptors (targeted by propfol and etomidate), NMDA receptors (targeted by ketamine), or Alpha2 adrenergic receptors (targeted by dexmedetomidine). These data suggest that WB3 works to induce anesthetic effects through a different mechanism than other SOAs. Although no high-affinity targets were identified through these studies, certain enzymes from the cytochrome (CYP) P450 family were identified as potential targets. Accordingly, to further assess whether the identified CYPs were targets of WB3, tadpoles were treated with various inhibitors for CYP2C9, CYP3A5, and CYP26B1. Inhibition of CYP2C9 or CYP26B1 with talarozole alone did not mimic the effects observed with WB3 treatment (FIGS. 20A and 20B). Similarly, inhibition of CYP2C9 or CYP3A5 alone did not affect movement, but concurrent inhibition of CYP2C9 and CYP3A4 showed some similarities to WB3 at 100 μM.

| Inhibitory Target | Compound |
|---|---|
| CYP2C9 | APD668, Bucolome (Buc) |
| CYP3A5 | Clobetasol propionate (Clo) |
| CYP2C9 and CYP3A4 | Tetrahydrocurcumin (Tet), Apigenin (Api) |

### Mice Studies:

A formulation of WB3 in 25% PEG300 and saline was used for intraperitoneal (IP) administration in mice. No effects were observed with injection of vehicle alone. Accordingly, IP injections of 10, 30, 50, 100, 150, 200 mg/kg of WB3 were formulated in 25% PEG300. A slowing of movement was observed at concentrations equal to or greater than 100 mg/kg in a dose dependent manner. No signs of pain or distress were observed. The largest effect was observed at 200 mg/kg IP, which lasted more than 1.5 hour. All animals fully recovered. The same formulation was injected intravascular (IV) at 10, 30 and 100 mg/kg. Hyperactivity and increased breathing were observed at 10 mg/kg and 30 mg/kg for a few minutes (<5 min).

### EQUIVALENTS AND SCOPE

In the claims articles such as “a.” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A method of inducing anesthesia in a subject in need thereof, the method comprising administering a compound of Formula (1): 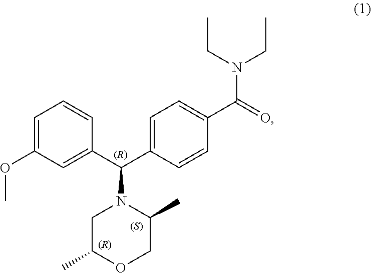
or a pharmaceutically acceptable salt, solvate, hydrate, polymorph, co-crystal, tautomer, stereoisomer, isotopically labeled derivative, or prodrug thereof to the subject.

2. The method of claim 1, wherein the compound is administered in a pharmaceutical composition comprising one or more pharmaceutically acceptable excipients.

3. The method of claim 1, wherein the compound, or a pharmaceutical composition comprising the compound, is administered orally, topically, intramuscularly, intravenously, or parenterally.

4. The method of claim 1, wherein the compound is administered at a dose sufficient to achieve a desired anesthetic effect, wherein the desired anesthetic effect is selected from the group consisting of general anesthesia, sedation, tranquilization, immobility, amnesia, analgesia, unconsciousness, and autonomic quiescence.

5. (canceled)

6. The method of claim 4 or 5, wherein the desired anesthetic effect is general anesthesia.

7. The method of claim 1, wherein inducing anesthesia in a subject comprises physiological slowing in the subject, wherein physiological slowing comprises slowing of metabolic processes in the subject.

8. (canceled)

9. The method of claim 1, wherein inducing anesthesia is reversible in the subject.

10. The method of claim 9, wherein reversing induction of anesthesia comprises restoration of mobility and/or consciousness in the subject.

11. The method of claim 1, wherein the subject is undergoing surgery and/or a medical procedure.

12. The method of claim 1, wherein the subject is in pain and/or is injured.

13. The method of claim 1, wherein the subject is a mammal.

14. The method of claim 13, wherein the mammal is a human.

15. A method of treating pain in a subject in need thereof, the method comprising administering a compound of Formula (1): 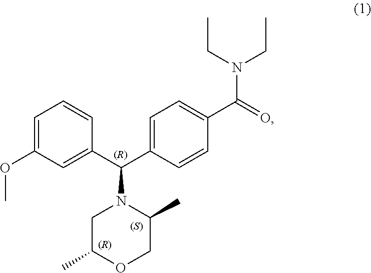
or a pharmaceutically acceptable salt, solvate, hydrate, polymorph, co-crystal, tautomer, stereoisomer, isotopically labeled derivative, or prodrug thereof to the subject.

16. The method of claim 15, wherein the compound is administered in a pharmaceutical composition comprising one or more pharmaceutically acceptable excipients.

17. The method of claim 15, wherein the compound, or a pharmaceutical composition comprising the compound, is administered orally, topically, intramuscularly, intravenously, or parenterally.

18. The method of claim 15, wherein the pain is nociceptive pain, neuropathic pain, or a combination thereof.

19. The method of claim 15, wherein the pain is acute pain, chronic pain, mild pain, moderate pain, or severe pain.

20. The method of claim 15, wherein administering the compound of Formula (1) does not induce general anesthesia, sedation, tranquilization, immobility, amnesia, unconsciousness, and/or autonomic quiescence.

21. The method of claim 15, wherein the subject is a mammal.

22. The method of claim 21, wherein the mammal is a human.

23-25. (canceled)
